(19) United States
(12) Patent Application Publication
(10) Pub. No.: US 2024/0307427 A1
(43) Pub. Date: Sep. 19, 2024

(54) REMDESIVIR COCRYSTAL, COMPOSITIONS AND METHODS THEREOF

(71) Applicants: VERSITECH LIMITED, Hong Kong (CN); Advanced Biomedical Instrumentation Centre Limited, Hong Kong (CN)

(72) Inventors: Shing Fung Chow, Hong Kong (CN); Kam Hung Low, Hong Kong (CN); Si Nga Wong, Hong Kong (CN)

(73) Assignees: VERSITECH LIMITED, Hong Kong (CN); Advanced Biomedical Instrumentation Centre Limited, Hong Kong (CN)

(21) Appl. No.: 18/594,986

(22) Filed: Mar. 4, 2024

Related U.S. Application Data
(60) Provisional application No. 63/489,894, filed on Mar. 13, 2023.

(51) Int. Cl. A61K 31/706; A61K 9/00; A61K 31/60

(52) U.S. Cl. CPC A61K 31/706; A61K 9/0075; A61K 31/60

(57) ABSTRACT

Provided is a cocrystals of remdesivir (RDV) composed of a 1:1 molar ratio of RDV and benzoic acid derivative. The benzoic acid derivative can be salicylic acid (SA). The RDV-SA cocrystal are formed by either liquid-assisted grinding or spray-drying, followed by thermal annealing to facilitate cocrystal formation. The RDV-SA cocrystals can be formulated as inhalable dry powders and included in a medicament for use in treatments for influenza viral infections, such as COVID-19. The inhalable RDV-SA cocrystal dry powders is suitable for deep lung delivery, with good dissolution performance.

### CROSS REFERENCE

This application claims the benefit of U.S. Provisional Patent Application No. 63/489,894, filed on Mar. 13, 2023, which is incorporated by reference in its entirety.

### 1. FIELD

The present disclosure relates to a remdesivir cocrystal, its composition, methods of making and therapeutic uses thereof.

### 2. BACKGROUND

Remdesivir (RDV) is an adenosine nucleotide analog which exhibits broad-spectrum activities against RNA viruses. Intravenous RDV is one of the approved medications for the treatment of COVID-19, yet is unsuitable for treatment in outpatient settings as trained healthcare professionals are required, associated with high indirect medical cost and low patient compliance. It is also contraindicated in patients with severe renal impairment due to the presence of sulfobutylether-beta-cyclodextrin as a solubility enhancer. These highlight the need to develop next-generation RDV formulations which can be widely deployed to outpatients. While oral administration of RDV is preferred, a significant portion of RDV is extracted by the liver such that only a small fraction could reach the lungs, i.e., the primary site of COVID-19 infection and converted into its active triphosphate metabolite to exert its antiviral effect. Its erratic aqueous solubility (<0.03 mg/mL) also hinders its absorption into the circulation. This may render RDV less competitive than other oral marketed antivirals such as molnupiravir and nirmatrelvir/ritonavir. A prodrug strategy has led to the identification of an orally bioavailable RDV analog (GS-621763) with antiviral efficacy in animal models, whereas its clinical efficacy remains unknown.

### 3. SUMMARY

The present disclosure demonstrates the use of a simple but useful strategy to access hidden cocrystals. Co-grinding of RDV and SA followed by high temperature annealing in solid state allows the coformers to swiftly reach the “activated” state, where the rearrangement of molecules into cocrystal is possible. As the optimal temperatures for thermal annealing usually do not match the ambient conditions, there exist abundant hidden cocrystals being trapped in amorphous intermediate phase during the historical cocrystal screening journey, but unfortunately could not be unveiled due to the lack of precise control on the annealing conditions. The present disclosure provides a low cost thermally activated cocrystallization process in expanding the solid-state diversity of pharmaceuticals.

The inventors discovered that RDV which mimics the structural feature of other antivirals, such as acyclovir, abacavir, telaprevir, and ribavirin, can form cocrystals via O—H . . . N and C—O . . . H interactions between the 4-aminopyrrolotriazine moiety of RDV and the carboxylic acid group of the selected acid. The inventors developed in one embodiment, an inhalable RDV cocrystal-based dry powder formulation for COVID-19 treatment via cocrystal engineering. In certain embodiments, the present disclosure: a) synthesized the first RDV cocrystal with BA derivatives as coformers via liquid assisted grinding (LAG) and spray drying, followed by solid-state characterization and structural determination of the resulting RDV cocrystal; (b) provides different processing parameters of cocrystal fabrication (e.g., the nature of organic solvents, annealing temperature) that affect the RDV cocrystallization outcomes; and (c) optimized RDV cocrystal-based dry powder formulation with regards to the aerosol performance and in vitro cytotoxicity.

The inventors developed sophisticated cocrystal-based formulations, through advanced manipulation of cocrystal and particle engineering of inhaled pharmaceuticals.

In one embodiment, provided is a cocrystal of remdesivir and salicylic acid.

In one embodiment, the cocrystal belongs to a monoclinic P2₁ space group and having unit cell parameters a=18.3±0.1 Ang, b=5.6±0.1 Ang, c=19.3±0.1 Ang, α=γ=90°, β=112±1°.

In one embodiment, the cocrystal is characterized by a powder X-ray diffraction (PXRD) pattern measured using an incident beam of Cu Ka radiation comprising at least two X-ray diffraction peaks and the corresponding d-spacing values substantially as shown in Table 4.

In one embodiment, the cocrystal is characterized by a powder X-ray diffraction (PXRD) pattern measured using an incident beam of Cu Ka radiation comprising X-ray diffraction peaks below 20 degree two-theta substantially as shown in Table 4.

In one embodiment, the cocrystal is characterized by a powder X-ray diffraction (PXRD) pattern measured using an incident beam of Cu Ka radiation comprising X-ray diffraction peaks substantially as shown in Table 4.

In one embodiment, the cocrystal is characterized by a powder X-ray diffraction (PXRD) pattern measured using an incident beam of Cu Ka radiation comprising X-ray diffraction peaks below 20 degree two-theta substantially as shown in FIG. 7A (top 5 panels) or 7B (top 5 panels).

In one embodiment, the cocrystal is characterized by a powder X-ray diffraction (PXRD) pattern measured using an incident beam of Cu Ka radiation comprising X-ray diffraction peaks substantially as shown in FIG. 7A (top 5 panels) or 7B (top 5 panels).

In one embodiment, the cocrystal is characterized by a powder X-ray diffraction (PXRD) pattern measured using an incident beam of Cu Ka radiation substantially as shown in FIG. 7B (top five panels) or Table 4.

In one embodiment, the cocrystal is characterized by a Differential Scanning calorimetry (DSC) profile having a melting endotherm at about 138° C. to about 160° C., preferably at about 150° C. to about 155° C.

In one embodiment, the cocrystal is characterized by a Differential Scanning calorimetry (DSC) profile substantially as shown in FIG. 4B (top five panels) or FIG. 10.

In one embodiment, the cocrystal is characterized by Fourier-Transform Infrared Spectroscopy (FTIR) profile substantially as shown in FIG. 14.

In certain embodiments, the RDV−SA cocrystal is prepared by the following methods:
1) liquid-assisted grinding (LAG) followed by thermal annealing for cocrystal screening; or
2) Spray drying followed by thermal annealing for developing inhalable dry powder formulation.

In one embodiment, provided is a cocrystal prepared by a process comprising (a) liquid-assisted grinding; and (b) thermal annealing.

In one embodiment, provided is a cocrystal prepared by a process comprising (a) spray drying; and (b) thermal annealing.

In one embodiment, provided is a process for preparing a cocrystal of remdesivir and salicylic acid, the process comprising the steps of:
(a) grinding remdesivir and salicylic acid in the presence of a solvent to produce a mixture; and
(b) thermally annealing the mixture of step (a) at a temperature of at least about 60° C., preferably at least about 80° C.

In one embodiment, provided is a process for preparing a powder for inhalation, the powder comprising a cocrystal of remdesivir and salicylic acid, the process comprising the steps of:
(a) spray drying a solution of remdesivir and salicylic acid in a solvent to produce a mixture; and
(b) thermally annealing the mixture of step (a) at a temperature of at least about 60° C., preferably at least about 80° C.

In certain embodiments, the solvent is selected from the group consisting of acetonitrile, ethanol, methanol, acetone, and chloroform.

In one embodiment, provided is a pharmaceutical composition comprising as an active ingredient a remdesivir cocrystal according to the present disclosure. In one embodiment, the composition is in a form of a powder for inhalation. In some embodiments, the powder for inhalation is excipient free. In some embodiments, the powder for inhalation comprises one or more excipients.

In some embodiments, the composition has a median mass aerodynamic diameter (MMAD) of below about 5 microns. In some embodiments, the composition has a median mass aerodynamic diameter (MMAD) between about 1 and about 5 microns. In some embodiments, the composition has a MMAD of 4.33±0.2 μm.

In some embodiments, the composition comprises a fine particle fraction (FPF) of at least about 30%. In some embodiments, the composition comprises a fine particle fraction (FPF) of 41.39±4.25%. In one embodiment, provided is a method of treating a respiratory viral infection in a subject in need thereof, comprising administering to the respiratory system of the subject a composition comprising a therapeutically-effective amount of a cocrystal of remdesivir and salicylic acid. In one embodiment, provided is a cocrystal of remdesivir and a coformer having a molar ratio of 1:1.

In some embodiments, the coformer comprises at least one functional group selected from the group consisting of carboxylic acid, hydroxy, ether, aldehyde, ketone, ester, amide, amine, phosphonic acid, and sulfonic acid.

In some embodiments, the coformer comprises a benzoic acid derivative.

In some embodiments, the coformer is selected from the group consisting of salicylic acid, acetylsalicylic acid, and gentisic acid

In one embodiment, provided is a method of treating a respiratory viral infection in a subject in need thereof, comprising administering to the respiratory system of the subject a composition comprising a therapeutically-effective amount of a cocrystal of remdesivir and a coformer.

In some embodiments, the respiratory virus is a coronavirus. In some embodiments, the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus. In some embodiments, the respiratory virus is COVID-19.

In some embodiments, the composition is administered by oral inhalation. In some embodiments, the composition is administered by nasal inhalation. In some embodiments, the composition is administered with a dry powder inhaler.

### 4. BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 Chemical structures of RDV and the selected benzoic acid derivative coformers.

FIG. 2. Thermal analysis of the 1:1 RDV-BA, RDV−SA, RDV-ASA, and RDV-GA systems produced by liquid-assisted grinding prior to thermal annealing.

FIG. 3. Thermal analysis of the 1:1 RDV-BA, RDV-ASA, and RDV-GA systems produced by liquid-assisted grinding after thermal annealing.

FIGS. 4A-B. Thermal analysis of the 1:1 RDV−SA cocrystal systems produced by liquid-assisted grinding in different solvent systems, annealed at (A) room temperature and (B) 80° C.

FIG. 5. Temperature-composition phase diagram of the RDV−SA cocrystal system.

FIGS. 6A-C. DSC thermograms of RDV−SA obtained by LAG in A) ACN and B) EtOH prior to thermal annealing, and C) pure RDV powders obtained by spray drying. The midpoint of the glass transition was recorded as the glass transition temperature (T_{g}), indicated by the arrows.

FIGS. 7A-B. PXRD patterns of RDV−SA produced by liquid-assisted grinding in different solvent systems, annealed at (A) room temperature and (B) 80° C.

FIG. 8. Thermal annealing at high temperature only and without co-grinding of the physical mixtures of RDV and SA failed to produce phase pure RDV−SA cocrystals, as indicated by the DSC thermogram.

FIG. 9. Thermal analysis of the LAG RDV−SA at different annealing times at 60° C. and 80° C.

FIG. 10. Thermal analysis of RDV−SA produced by spray drying (SD) in acetonitrile, annealed at room temperature and 80° C., respectively.

FIG. 11. PXRD patterns of RDV−SA produced by spray drying (SD) in acetonitrile, annealed at room temperature and 80° C., respectively.

FIG. 12. The ¹³C NMR spectrum in a d₄-methanol solution of a 1:1 mixture of RDV and SA (upper) and the solid-state ¹³C CP-MAS NMR spectrum of the thermally annealed LAG RDV−SA cocrystal (lower). Molecular structures with atom numbers of RDV and SA are shown with the phenolic and pyrrolotriazine ring carbon atoms of RDV in orange and blue, respectively, and those carbon atoms of SA in green for clarity. Resonance peak assignments were achieved by comparison with the reported values of the ribonucleoside GS-441524 and the individual ¹³C NMR spectra of the cocrystal component molecules.

FIGS. 13A-B. (A) Crystal structure of the 2×2×1 unit cell of the thermally annealed LAG RDV−SA cocrystal system viewed along the b-axis. Hydrogen atoms are not shown for clarity. (B) Illustration of Intermolecular hydrogen bondings between RDV and SA molecules with blue dash lines.

FIG. 14. FTIR spectra of the RDV−SA cocrystal system produced by liquid-assisted grinding in acetonitrile.

FIGS. 15A-C. NGI dispersion data of spray dried RDV−SA formulations (n=3). S1-S7 presents impactor stages 1-7, followed by the corresponding upper aerodynamic cutoff diameter in parentheses. MOC is the micro-orifice collector in the NGI. (A) SD1 (B) SD2 (C) SD3.

FIG. 16. Dissolution profiles of RDV, RDV−SA, and physical mixture of RDV and SA (RDV+SA) in pH 7.4 simulated lung fluid (n=3).

FIGS. 17A-B. Real-time pH values against time during the dissolution of RDV, SA, and RDV−SA.

FIG. 18. Cell viability of the raw drugs, the physical mixture and the RDV−SA cocrystal at concentrations from 0.05 to 10 μM.

FIG. 19. Illustration of an embodiment of the process of developing inhalable RDV−SA cocrystal DPI.

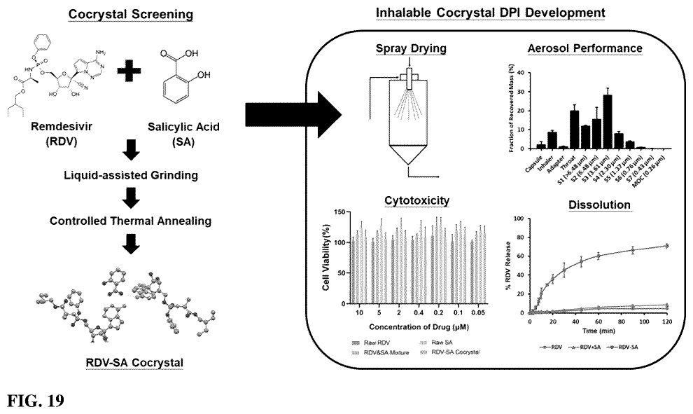

FIG. 20. TGA profiles of the RDV−SA cocrystal systems produced using different organic solvents by LAG and spray drying.

FIG. 21. HPLC chromatograms indicating the stability assessment of RDV in LAG RDV−SA under the thermal annealing treatment at 80° C.

FIGS. 22A-B. Thermal analysis of the LAG RDV−SA produced in ACN at different grinding times (A) before and (B) after thermal annealing at 80° C.

FIG. 23. Scanning electron micrographs of spray-dried RDV (SD_RDV), and spray-dried RDV−SA cocrystal powders (SD1, SD2, SD3) at 5000× magnification.

### 5. DETAILED DESCRIPTION

Pulmonary delivery of RDV is a propitious approach to facilitate carly treatment to outpatients, while simultaneously providing targeted therapy to the respiratory tract. Inhalation of aerosols can maintain a high drug concentration in the inflamed lung endothelial cells and result in rapid onset of action for viral eradication and enhanced therapeutic efficacy, making dose reduction possible. In addition, the respiratory tract provides a less harsh and low cytochrome P450 enzymatic environment which minimizes conversion of RDV into inactive oxidized metabolites, particularly useful for improving the efficacy of RDV.

Efficient activation of RDV also occurs in human lung cells through intracellular metabolism of RDV and its alanine intermediate (MetX) to monophosphate and successive phosphorylation to form the active nucleoside triphosphate GS-443902, facilitating an effective treatment of respiratory viral infections such as COVID-19.

### 5.1 Formulations

In some embodiments, inhaled RDV can be beneficial in reduction of viral burden. In some embodiments, the RDV−SA cocrystal of the present disclosure can be administered by oral inhalation. In some embodiments, the RDV−SA cocrystal of the present disclosure can be administered by nasal inhalation. In some embodiments, the RDV−SA cocrystal of the present disclosure can be administered in a nasal spray. In some embodiments, the RDV−SA cocrystal of the present disclosure can be administered in a nasal mist. In some embodiments, the RDV−SA cocrystal of the present disclosure can be administered with a dry powder for inhalation.

In some embodiments, the RDV−SA cocrystal of the present disclosure can be administered with or as a metered dose inhaler (MDI), nebulizer, soft mist inhalation, a nasal aerosol, a propellant-containing metered-dose aerosol, an inhalable solution or inhalable suspension, a propellant-free inhalable solution, a propellant-free inhalable suspension, and the like.

In some embodiments, the RDV−SA cocrystal of the present disclosure can be with a dry powder inhaler (DPI). Among different inhalation devices specifically designed for generating drug aerosols, dry powder inhalers (DPIs) are gaining popularity thanks to their propellant-frec and portable nature, which utilize the patient's inspiratory flow for aerosol dispersion and entrainment into the lungs. Apart from case of administration, pulmonary delivery of DPI offers several merits such as better stability compared with liquid formulations, and thus delivering drugs in high potency that leads to more effective treatment of lung infections, particularly for antiviral and antimicrobial purposes. The risk of contamination and transmission of pathogenic particles caused by the application of nebulizer can also be mitigated. Despite DPI being an appealing localized treatment approach for lung diseases, the application of drugs with low aqueous solubility is often restricted because of the dissolution obstacles in a small volume of lining fluid in the lungs.

In some embodiments, the powder for inhalation is excipient free. In some embodiments, the powder for inhalation comprises one or more excipients. In some embodiments, the pharmaceutical composition is excipient free. In some embodiments, the pharmaceutical composition comprises one or more excipients.

Suitable pharmaceutically-acceptable excipients include, but are not limited to, a solvent, a thickening agent, a plasticizer, a preservative, an absorption or permeability enhancing agent, a conditioner, an emulsifier, a surfactant, an antioxidant, a buffering agent, an emollient, a humectant, a suspending agent, an opacifier, and a binder.

Thickening agents include, but are not limited to hydroxyethyl cellulose, microcrystalline cellulose (MCC) and sodium carboxymethyl cellulose.

Plasticizers include, but are not limited to caprylic acid, capric triglyceride and triethyl citrate.

Binders include, but are not limited to microcrystalline cellulose, gum tragacanth, gum arabic, gelatin, polyvinylpyrrolidone, copovidone, hydroxypropyl methylcellulose, and starch.

Surfactants include, but are not limited to sodium laurylsulfate or polysorbates, polyvinyl alcohol (PVA), polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil, and hardened castor oil such as polyoxyethylene hardened castor oil.

Preservatives include, but are not limited to benzalkonium chloride, benzoxonium chloride, thiomersal, phenylmercuric nitrate, phenylmercuric acetate, phenylmercuric borate, methylparaben, propylparaben, chlorobutanol, benzyl alcohol, and phenyl alcohol,

Antioxidants include, but are not limited to vitamins, provitamins, ascorbic acid, vitamin E or salts or esters thereof.

Absorption or permeability enhancing agent include, but are not limited to alkylglycosides, benzalkonium chloride, oleic acid, or salt thereof, polysorbate 20, polysorbate 80, sodium lauryl sulfate, cyclodextrins, medium and long chain fatty acids, or salts thereof, saturated and unsaturated fatty acids, or salts thereof, alcohol, glycerin, propylene glycol, PEG 300/400, and benzyl alcohol.

Buffering agents include, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.11% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.11% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, between about 0.1% and about 1%, by mass of the formulation.

In some embodiments, the composition has a median mass aerodynamic diameter (MMAD) of below about 5 microns. In some embodiments, the composition has a median mass aerodynamic diameter (MMAD) of below about 4 microns. In some embodiments, the composition has a median mass aerodynamic diameter (MMAD) of below about 3 microns. In some embodiments, the composition has a median mass acrodynamic diameter (MMAD) of below about 2 microns. In some embodiments, the composition has a median mass aerodynamic diameter (MMAD) of below about 1 micron. In some embodiments, the composition has a median mass aerodynamic diameter (MMAD) between about 1 and about 5 microns. In some embodiments, the composition has a median mass aerodynamic diameter (MMAD) between about 2 and about 5 microns. In some embodiments, the composition has a median mass acrodynamic diameter (MMAD) between about 3 and about 5 microns. In some embodiments, the composition has a median mass aerodynamic diameter (MMAD) between about 4 and about 5 microns. In some embodiments, the composition has a median mass aerodynamic diameter (MMAD) between about 4 and about 4.5 microns. In some embodiments, the composition has a median mass aerodynamic diameter (MMAD) between about 4.5 and about 5 microns. In some embodiments, the composition has a MMAD of 4.33±0.2 μm.

In some embodiments, the composition comprises a fine particle fraction (FPF) of at least about 30%. In some embodiments, the composition comprises a fine particle fraction (FPF) of at least about 35%. In some embodiments, the composition comprises a fine particle fraction (FPF) of at least about 40%. In some embodiments, the composition comprises a fine particle fraction (FPF) of at least about 45%. In some embodiments, the composition comprises a fine particle fraction (FPF) of at least about 50%. In some embodiments, the composition comprises a fine particle fraction (FPF) between about 30% and about 50%. In some embodiments, the composition comprises a fine particle fraction (FPF) between about 30% and about 35%. In some embodiments, the composition comprises a fine particle fraction (FPF) between about 35% and about 40%. In some embodiments, the composition comprises a fine particle fraction (FPF) between about 40% and about 45%. In some embodiments, the composition comprises a fine particle fraction (FPF) between about 35% and about 40%. In some embodiments, the composition comprises a fine particle fraction (FPF) of 41.39±4.25%. In some embodiments, the composition comprises a fine particle fraction (FPF) of 41.39±4.25% w/w.

### 5.2 RDV Polymorphs

There are four known polymorphic forms of RDV (Forms I, II, III, and IV). Form I and Form II exhibit differences in physicochemical properties and pharmacokinetics, while Form III and Form IV are poorly characterized. RDV Form I has a marginally faster dissolution rate than RDV Form II, but still is insufficient to provide an adequate therapeutic response. Notably, cocrystallization constitutes an untapped strategy for expanding the solid-state landscape of RDV and simultaneously improving its pharmaceutical properties, such as solubility, dissolution, stability, hygroscopicity, and aerosol performance, etc. Combining the merits of cocrystallization and particle engineering via spray drying also propels the development of excipient-free DPI formulation, negating concerns about the safety and tolerability of excipients from a regulatory perspective.

Any one of the aforementioned polymorphs can be incorporated into the cocrystal of the present disclosure. In some embodiments, the present disclosure provides a remdesivir cocrystal with salicylic acid, wherein the remdesivir is Form I. In some embodiments, the present disclosure provides a remdesivir cocrystal with salicylic acid, wherein the remdesivir is Form II. In some embodiments, the present disclosure provides a remdesivir cocrystal with salicylic acid, wherein the remdesivir is Form III. In some embodiments, the present disclosure provides a remdesivir cocrystal with salicylic acid, wherein the remdesivir is Form IV.

### 5.3 RDV Cocrystals

The term “cocrystal” refers to a molecular complex derived from an association of a drug with a coformer. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

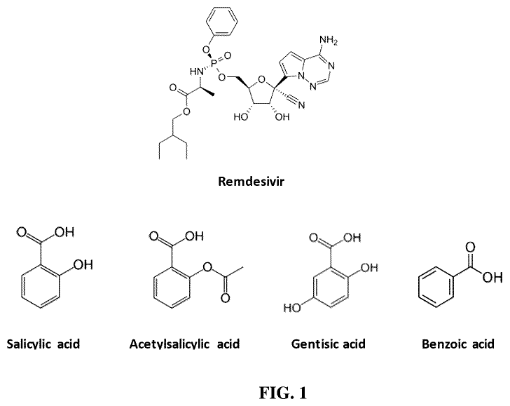

No RDV cocrystal has been discovered to date. Identifying proper coformers interacting with the drug molecule from the point of view of supramolecular chemistry is essential for successful cocrystal screening. Benzoic acid (BA) and its derivatives, namely salicylic acid (SA), acetylsalicylic acid (ASA), and gentisic acid (GA) were selected as the coformers in the present disclosure (FIG. 1).

In one embodiment, provided is a cocrystal of remdesivir and a coformer. In one embodiment, provided is a cocrystal of remdesivir and a coformer having a molar ratio of 1:1.

In some embodiments, the coformer comprises at least one functional group selected from the group consisting of carboxylic acid, hydroxy, ether, aldehyde, ketone, ester, amide, amine, phosphonic acid, and sulfonic acid.

In some embodiments, the coformer comprises a benzoic acid derivative.

In some embodiments, the coformer is salicylic acid. In some embodiments, the coformer is acetylsalicylic acid. In some embodiments, the coformer is gentisic acid.

Mechanochemical grinding and rapid solution evaporation of drugs could readily generate amorphous materials. Thermal annealing facilitates the diffusion and crystal lattice rearrangement, eventually leading to drug recrystallization or the formation of new drug polymorphs. In the context of cocrystal, this kind of thermally activated “on-off” control on phase transformation was commonly seen in metal cocrystals for safety improvement, while those related to the discovery of organic cocrystals is limited. The annealing assisted mechanochemical syntheses of transition-metal coordination compounds and cocrystal formation at high temperature demonstrates that heating of solid reactants could be a useful approach for the solid-state synthesis in metal complex chemistry. A rational cocrystal screening should take into account the optimization of annealing conditions, especially for compounds with high molecular weight and glass forming ability (GFA). Certain elusive pharmaceutical cocrystals could only form under controlled annealing conditions, which normally do not match the ambient conditions, representing a pitfall in cocrystal screening.

Despite advances in knowledge-based cocrystal design, it is difficult to efficiently and logically identify coformer pairs with complementary functional groups e.g., based on Hansen solubility parameters, supramolecular synthons, structural resemblance. The emergence of in silico virtual screening via evaluating molecular electrostatic potential surfaces and hydrogen bond propensity have greatly increased the overall hit rate, however it is still not uncommon to obtain false positive results (with predicted cocrystals but not experimentally observed). Although false positives are less of a concern than false negatives, it remains obscure that the molecules containing functional groups indicating a high likelihood of being successful coformers would eventually fail. The present disclosure provides a low cost thermally activated cocrystallization process for development of remdesivir cocrystal.

In one embodiment, provided is a cocrystal of remdesivir and salicylic acid.

In one embodiment, the cocrystal belongs to a monoclinic P2₁ space group and having unit cell parameters a=18.3±0.1 Ang, b=5.6±0.1 Ang, c=19.3±0.1 Ang, α=γ=90°, β=112±1°.

In one embodiment, the cocrystal is characterized by a powder X-ray diffraction (PXRD) pattern measured using an incident beam of Cu Ka radiation comprising at least two X-ray diffraction peaks and the corresponding d-spacing values substantially as shown in Table 4.

In one embodiment, the cocrystal is characterized by a powder X-ray diffraction (PXRD) pattern measured using an incident beam of Cu Ka radiation comprising X-ray diffraction peaks below 20 degree two-theta substantially as shown in Table 4.

In one embodiment, the cocrystal is characterized by a powder X-ray diffraction (PXRD) pattern measured using an incident beam of Cu Ka radiation comprising X-ray diffraction peaks substantially as shown in Table 4.

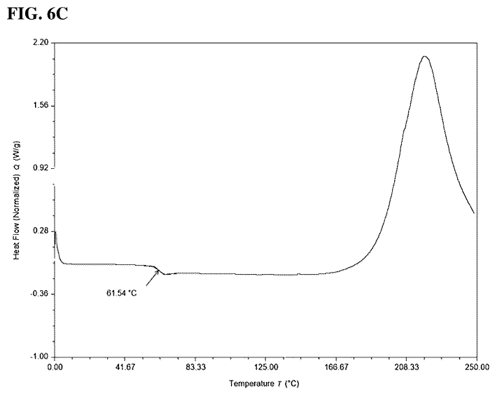

In one embodiment, the cocrystal is characterized by a powder X-ray diffraction (PXRD) pattern measured using an incident beam of Cu Ka radiation comprising X-ray diffraction peaks below 20 degree two-theta substantially as shown in FIG. 7A (top 5 panels) or 7B (top 5 panels).

In one embodiment, the cocrystal is characterized by a powder X-ray diffraction (PXRD) pattern measured using an incident beam of Cu Ka radiation comprising X-ray diffraction peaks substantially as shown in FIG. 7A (top 5 panels) or 7B (top 5 panels).

In one embodiment, the cocrystal is characterized by a powder X-ray diffraction (PXRD) pattern measured using an incident beam of Cu Ka radiation substantially as shown in FIG. 7A (top five panels) or Table 4.

In one embodiment, the cocrystal is characterized by a powder X-ray diffraction (PXRD) pattern measured using an incident beam of Cu Ka radiation substantially as shown in FIG. 7B (top five panels) or Table 4.

In one embodiment, the cocrystal is characterized by a powder X-ray diffraction (PXRD) pattern having at least two X-ray diffraction peaks at 5.14±0.1, 5.62±0.1, 8.32±0.1, 15.00±0.1, 16.52±0.1, 18.58±0.1, and 21.48±0.1 degrees two theta (°□).

In one embodiment, the cocrystal is characterized by a powder X-ray diffraction (PXRD) pattern having X-ray diffraction peaks at 5.14±0.1, 5.62±0.1, 8.32±0.1, 15.00±0.1, 16.52±0.1, 18.58±0.1, and 21.48±0.1 degrees two theta (°□).

In one embodiment, the cocrystal is characterized by a Differential Scanning calorimetry (DSC) profile having a melting endotherm at about 138° C. to about 160° C., preferably at about 150° C. to about 155° C.

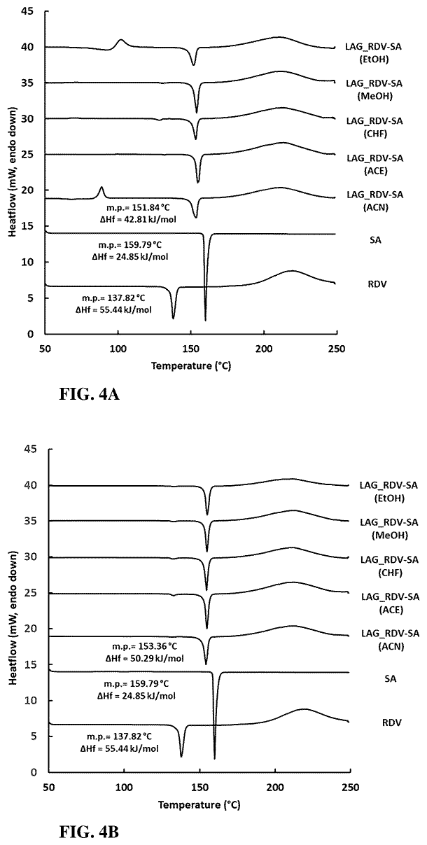

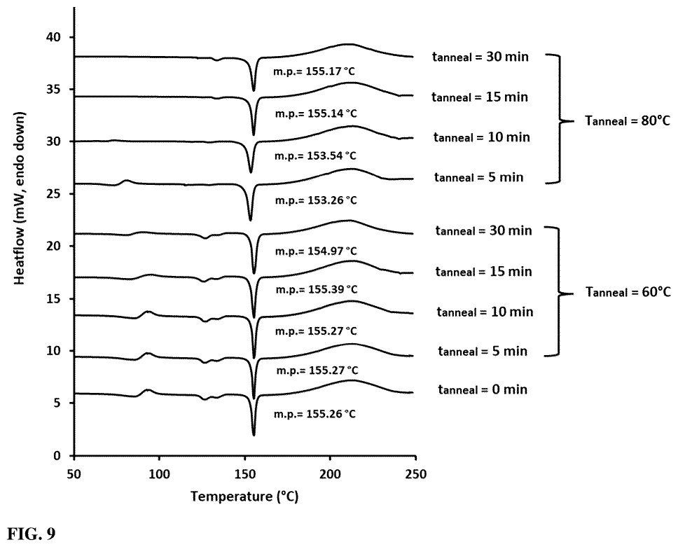

In one embodiment, the cocrystal is characterized by a Differential Scanning calorimetry (DSC) profile substantially as shown in FIG. 4B (top five panels) or FIG. 10.

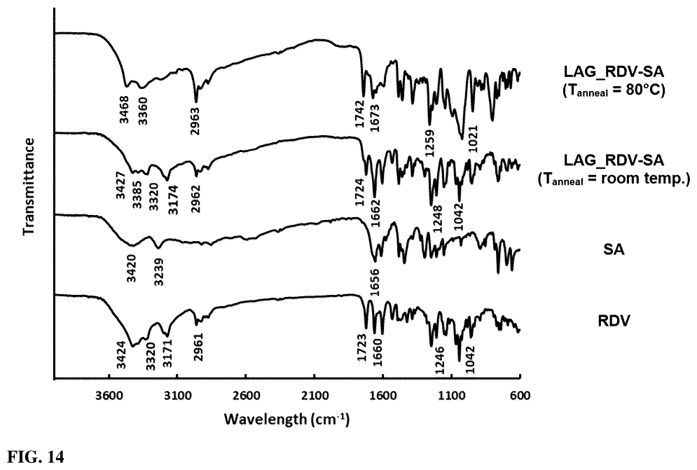

In one embodiment, the cocrystal is characterized by Fourier-Transform Infrared Spectroscopy (FTIR) profile substantially as shown in FIG. 14.

### 5.4 Therapeutic Uses

In the context of therapeutic applications, most of the selected coformers exhibit antiviral and anti-inflammatory properties which may synergize with RDV by means of cocrystallization for COVID-19 treatment. Notably, both SA and ASA can inhibit SARS-COV-2 viral replication in vitro in human precision-cut lung slices, which implies its potential to synergistically enhance the antiviral effect of RDV. Although inhalation of acidic coformers may arouse concerns for respiratory irritation, a very low dose (0.35 mg/kg once daily) of inhaled RDV was sufficient to induce antiviral effect. Thus, safety should not be a significant barrier in the clinical use of the inhalable formulation.

In one embodiment, provided is a method of treating a respiratory viral infection in a subject in need thereof, comprising administering to the respiratory system of the subject a composition comprising a therapeutically-effective amount of a cocrystal of remdesivir and salicylic acid. In one embodiment, provided is a cocrystal of remdesivir and a coformer having a molar ratio of 1:1.

In one embodiment, provided is a method of treating a respiratory viral infection in a subject in need thereof, comprising administering to the respiratory systero of the subject a composition comprising a therapeutically-effective amount of a cocrystal of remdesivir and a coformer.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof.

In some embodiments. the respiratory virus is a coronavirus. In some embodiments, the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus. In some embodiments, the respiratory virus is COVID-19.

In some embodiments, the composition is administered by oral inhalation. In some embodiments, the composition is administered by nasal inhalation. In some embodiments, the composition is administered with a dry powder inhaler.

### 5.5 Methods of Preparing Cocrystals

In certain embodiments, the RDV−SA cocrystal is prepared by the following methods:
1) Liquid assisted grinding (LAG) followed by thermal annealing for cocrystal screening; or
2) Spray drying followed by thermal annealing for developing inhalable dry powder formulation.

In one embodiment, provided is a cocrystal prepared by a process comprising (a) liquid-assisted grinding; and (b) thermal annealing.

In one embodiment, provided is a cocrystal prepared by a process comprising (a) spray drying; and (b) thermal annealing.

In one embodiment, provided is a process for preparing a cocrystal of remdesivir and salicylic acid, the process comprising the steps of:
(a) grinding remdesivir and salicylic acid in the presence of a solvent to produce a mixture; and
(b) thermally annealing the mixture of step (a) at a temperature of at least about 60° C., preferably at least about 80° C.

In some embodiments, the annealing temperature can be at least about 60° C., about 65° C., about 70° C., about 75° C., about 80° C., about 85° C., about 90° C., about 95° C., about 100° C. or above.

Annealing can be carried out for a time period that is sufficient to form the RDV cocrystal of the present disclosure. In some embodiments, the annealing time is about 30 minutes. In some embodiments, the annealing time is about 30 minutes at a temperature of at least about 80° C. However, it is contemplated that longer or shorter time periods may be suitable, depending in part on the annealing temperature. For example, the cocrystal of the present disclosure can be formed at a lower annealing temperature to e.g., about 60° C., where the annealing is carried out for longer periods of time, for example about 60 minutes, about 90 minutes, about 120 minutes or even longer.

Exemplary annealing time periods are, e.g., about 10, 20, 30, 40, 50, 60, 70, 80, 90, 100, 110, 120 minutes, or even longer.

In one embodiment, provided is a process for preparing a powder for inhalation, the powder comprising a cocrystal of remdesivir and salicylic acid, the process comprising the steps of:
(a) spray drying a solution of remdesivir and salicylic acid in a solvent to produce a mixture; and
(b) thermally annealing the mixture of step (a) at a temperature of at least about 60° C., preferably at least about 80° C.

In certain embodiments, the solvent for liquid assisted drying is selected from the group consisting of polar organic solvents, including polar protic and polar aprotic solvents. Non-limiting examples of suitable solvents include lower alcohols, nitromethane, acetone, acetonitrile, ethyl acetate, dichloromethane, chloroform, dimethylformamide (DMF) and the like. In some embodiments, the solvent is selected from the group consisting of acetonitrile, ethanol, methanol, acetone, and chloroform.

### 6. EXAMPLES

In some embodiments, the present disclosure relates to a 1:1 cocrystal of remdesivir (“RDV”) and salicylic acid (“SA”) prepared from liquid assisted grinding (“LAG”). The clusiveness of the cocrystal is manifested by the thermally activated preparation method, where annealing at high temperature after grinding is essential to trigger the cocrystal growth. The use of different organic solvents as the cocrystallization medium in LAG generated different saturation levels, affecting the re-cocrystallization behavior of RDV−SA from an amorphous intermediate whereas the solvent effect was negated after annealing at high temperature. Compared to the raw RDV, the cocrystal displayed a superior dissolution performance in pH 7.4 simulated lung fluid and a good in vitro cytotoxicity profile in A549 cells. Spray drying was employed to reformulate the new RDV cocrystal for pulmonary delivery as inhalable dry powders, which can minimize the hepatic metabolism and directly target the infection site, potentially resulting in enhanced COVID-19 treatment. The optimized cocrystal formulation exhibited suitable MMAD and FPF for pulmonary delivery when dispersed at an inspiratory flow rate of 60 L/min. The favorable pharmaceutical properties of the cocrystal may be exploitable in the later formulation development and manufacturing of high-quality inhalable RDV products for clinical use. In certain embodiments, different annealing conditions are used during cocrystallization, particularly annealing temperature, in order to unveil more hidden cocrystals to facilitate the expansion of solid-state landscape.

In some embodiments, the present disclosure provides a successful synthesis of a cocrystal for remdesivir (RDV), an approved antiviral drug with broad-spectrum activities against RNA viruses. The RDV cocrystal was prepared with salicylic acid (SA) via combined liquid-assisted grinding (LAG) and thermal annealing. Formation of RDV−SA was found to be a thermally activated process, where annealing at high temperature after grinding was a prerequisite to facilitate the cocrystal growth from an amorphous intermediate, rendering it elusive under ambient preparing conditions. Through powder X-ray analysis with Rietveld refinement, the three-dimensional molecular structure of RDV−SA was resolved. The thermally annealed RDV−SA produced by LAG crystalized in a non-centrosymmetric monoclinic space group P2₁ with a unit cell volume of 1826.53(17) Å³, accommodating one pair of RDV and SA molecules in the asymmetric unit. The cocrystal formation was also characterized by differential scanning calorimetry, solid-state nuclear magnetic resonance, and Fourier-transform infrared spectroscopy. RDV−SA was further developed as inhaled dry powders by spray drying for potential COVID-19 therapy. The optimized RDV−SA dry powders exhibited a mass median acrodynamic diameter of 4.33±0.2 μm and fine particle fraction of 41.39±4.25% indicating the suitability for pulmonary delivery. Compared with the raw RDV, RDV−SA displayed a 15.43-fold higher fraction of release in simulated lung fluid at 120 min (p=0.0003). RDV−SA was safe in A549 cells without any in vitro cytotoxicity observed in the RDV concentration from 0.05 to 10 μM.

### 6.1 Materials

Remdesivir (RDV, ≥99%) form II was obtained from Yick Vic Chemicals & Pharmaceuticals Limited (Hong Kong SAR, China). Salicylic acid (SA, ≥99%) was purchased from Sigma-Aldrich (St. Louis, MO, USA). Ethanol and acetone of analytical grade were obtained from VWR BDH Chemicals (VWR International S.A.S., France). Methanol, acetonitrile, and chloroform of analytical grade were obtained from Merck KGAA (Darmstadt, Germany). Trifluoroacetic acid (TFA, ≥99%) was purchased from Sigma-Aldrich (St. Louis, MO, USA). Potassium bromide (KBr) for FTIR analysis was sourced from J&K Scientific Limited, China. Water was purified through a Thermolyne NANOpure Diamond Analytical ultra-pure water system (Barnstead, Thermo Fisher Scientific, Waltham, MA, USA). Dulbecco's Modified Eagle Medium:Nutrient Mixture F-12 (DMEM/F-12) cell culture medium, fetal bovine serum (FBS), antibiotic-antimycotic (100×), 0.25% (w/v) trypsin-EDTA solution, and 3-(4,5-dimethylthiazolyl-2)-2,5-diphenyltetrazolium bromide (MTT) were purchased from Thermo-Fisher Scientific (Waltham, MA, USA). A549 human alveolar epithelial adenocarcinoma cells were acquired from ATCC (Manassas, VA, USA).

### 6.2 Preparation of RDV Cocrystals

Attempts to cocrystallize RDV with BA, SA, ASA, and GA were first made using LAG. A 1:1 RDV−SA cocrystal was successfully obtained. The cocrystal was subsequently reproduced via spray drying for the purpose of inhaled dry powder formulation development.

### LAG

Equimolar amounts (0.405 mmol) of RDV form II (244.1 mg) and SA (55.9 mg) were gently ground with a mortar and pestle for 15 min at ambient conditions, with addition of 20 μl of ethanol (EtOH), methanol (MeOH), acetone (ACE), acetonitrile (ACN), or chloroform (CHF), respectively. The powders were frequently scraped out from the mortar and pestle, and re-mixed throughout the grinding process. The final products obtained from LAG were thermally annealed at 80° C. for 30 minutes in a laboratory oven (E23, BINDER Inc., New York, USA), and then gently triturated for further analysis. Prior to solid-state characterization, the variations in particle size and morphology were minimized by passing the samples through a standard test sieve with a mesh size of 63 μm (VWR International, New York, USA). The effects of different critical processing parameters on cocrystallization, including the solvent type, grinding time, annealing temperature, and annealing time, were investigated to identify the optimal conditions of LAG. As no discrepancy was observed among the samples produced from the selected solvents after thermal annealing, ACN with the highest solubility ratio, was used to reproduce the cocrystal using spray drying. Selection of ACN as the model solvent would allow a better observation on the difference in cocrystallization caused by the thermally activation process.

### Spray Drying

To produce inhalable cocrystal formulation, the solution containing equimolar amounts (0.405 mmol) of RDV form II (244.1 mg) and SA (55.9 mg) in ACN was spray-dried using a Büchi B-290 spray dryer with a standard two-fluid nozzle, glass chamber and a high-performance cyclone for collection of small particles (Büchi Labortechnik, Flawil, Switzerland). The spray dryer was equipped with a Buchi B-296 Dehumidifier and B-295 Inert Loop. The spray nozzle tip diameter was 0.7 mm. Nitrogen was used as the atomization gas. The RDV−SA DPI formulations were prepared under different solute concentrations, feed pump rates, and compressed gas atomization flow rates (Table 1), while inlet temperature and rate of aspirations were fixed at 60° C. and 100% (approximately 35 m³/h), resulting in an outlet temperature of 40-44° C. The final products were thermally annealed at 80° C. for 30 minutes in a laboratory oven (E23, BINDER Inc., New York, USA) prior to analysis.

TABLE 1
Aerodynamic size distribution (MMAD, GSD, and FPF) of spray-dried
RDV-SA formulations under different processing parameters.

| | TotalSolute | RDV | SA | Feed | Atomizing | | | |
|---|---|---|---|---|---|---|---|---|
| | concentration | Conc. | Conc. | pump rate | air flow | MMAD | GSD | FPF |
| Formulation | (mg/mL) | (mg/mL) | (mg/mL) | (mL/min) | (L/h) | (μm) | (μm) | (% w/w) |
| SD1 | 3 | 2.44 | 0.56 | 3 | 536 | 9.84 ± 0.41 | 2.72 ± 0.16 | 13.86 ± 1.41 |
| SD2 | 1.5 | 1.22 | 0.28 | 1.5 | 357 | 4.33 ± 0.20 | 2.00 ± 0.06 | 41.39 ± 4.25 |
| SD3 | 1.5 | 1.22 | 0.28 | 1.5 | 536 | 4.64 ± 0.23 | 1.82 ± 0.03 | 36.64 ± 5.21 |

### 6.3 Powder X-Ray Diffraction (PXRD) and Crystal Structure Determination

The PXRD pattern of RDV, SA and their cocrystal were collected by a Rigaku SmartLab 9 kW diffractometer with a copper rotating anode (K alpha1 1.54059 Å, K alpha2 1.54441 Å) rated at 200 mA/45 kV. Bragg Brentano CBO incident X-ray optics was used, with a 5.0 deg incident parallel Soller slit, a ½ degree incident slit, a 1.0×10.0 mm length limiting slit, a 5.0 deg receiving parallel slit, a ½ degree First receiving slit and a 0.3 mm second receiving slit. Diffraction signals were filtered with a K beta nickel filter, and diffraction data were collected with a HyPix-3000 detector in 1D mode. Possible unit cell parameters were obtained by N-TREOR (Version 2004) (Altomare et al., 2000) based on the diffraction pattern of the thermally annealed LAG RDV−SA. Unit cells with reasonable volumes were used for further analysis. Space group determination was performed by detecting the extinction group. The three-dimensional atomic coordinates of the individual components, RDV (Sekharan et al., 2021) and SA (Sundaralingam and Jensen, 1965), are available in the literature. These atomic coordinates were used as the initial models for the simulated annealing procedures in the EXPO2014 program suite (Altomare et al., 2013). Twenty-five simulated annealing experiments with ten structure solutions generated in each experiment were made for a consistent converging structure model. The structures with the lowest cost function were used for Rietveld refinement. All non-hydrogen atoms were refined isotropically. Bond length restraints were applied on the RDV and SA molecules according to the reported crystal structures of the individual compounds, while bond angle restraints were set according to CSD Mogul geometry search. Hydrogen atoms were included in the idealized positions. Select crystallographic results are provided in Table 6.

### 6.4 Solid-State and Solution Nuclear Magnetic Resonance Spectroscopy

Proton-decoupled Carbon-13 cross-polarisation magic-angle spinning (¹³C CP-MAS) measurements were carried out using a Bruker Avance III HD spectrometer (BRUKER BIOSPIN GmbH, Germany) and 4 mm (rotor o.d.) probe head with the Larmor frequency at 100.63 MHz. Spectra were acquired at a spin rate of 10 kHz. Cross-polarisation spectra were recorded with TOSS spinning sideband suppression, 1 ms contact time, and a recycle delay of 4 s. Carbon spectral referencing was relative to neat tetramethylsilane, carried out by setting the high-frequency signal from an external sample of adamantane to 38.5 ppm. Proton-decoupled ¹³C NMR solution nuclear magnetic resonance spectra were recorded in deuterated methanol (CD₃OD), with tetramethylsilane (TMS) as an internal standard on a Bruker Avance III HD spectrometer, operating with the Larmor frequency at 125.76 MHz. All spectra were calibrated with the solvent signals at δ49.00 ppm.

### 6.5 Thermal Analysis

Differential scanning calorimetry (DSC) and thermogravimetric analysis (TGA) profiles were generated by a TA DSC 250 differential scanning calorimeter (TA Instruments, New Castle, DE, USA) and a TGA Q5000 thermogravimetric analyzer (TA Company, New Castle, DE, USA), respectively. For SSC experiments indium was used for routine calibration of enthalpy and cell constant. An accurately weighed sample (˜3 mg) was encased in a Tzero Aluminum Hermetic pan (TA Instruments, New Castle, DE, USA) and heated from 50° C. to 250° C. at a scanning rate of 10° C./min. In the TGA experiments, each sample (˜3 mg mg) was placed on an open pan and heated at 10° C./min from 40° C. to 250° C. Nitrogen was used as the purge gas at 20 mL/min for both the DSC and TGA analyses. The TA Trios software was used for data analysis.

### 6.6 Fourier-Transform Infrared Spectroscopy (FTIR)

FTIR spectra were obtained using a FTIR spectrophotometer (Spectrum Two, PerkinElmer Instrument, USA) in a KBr diffuse reflectance mode. The scan was performed in the range of 4000 cm⁻¹ to 600 cm⁻¹ at an interval of 0.5 cm⁻¹. A total of 32 scans were performed at a resolution of 4 cm⁻¹ for each sample.

### 6.7 High Performance Liquid Chromatography (HPLC)

A high performance liquid chromatographic system (Agilent 1200 series, Agilent Technologies, Wilmington, DE, USA), equipped with a diode array detector and an Agilent Zorbax Eclipse Plus C18 column (5 μm, 250 mm×4.6 mm), was employed for quantitative analysis of RDV and SA in the solubility and drug release study. Samples were eluted at 1 mL/min under ambient conditions with a mobile phase composed of ACN and ultrapure water (45:55 v/v ratio) for RDV, and acetonitrile and 0.05% TFA (30:70 v/v ratio) for SA. 10 μL samples were injected onto the column. Detector was set at 244 and 230 nm for RDV and SA analyses, respectively.

### 6.8 In-Vitro Aerosol Performance Evaluation

The in vitro aerosol performance of the spray-dried RDV−SA cocrystal powder formulations was assessed with a Next Generation Impactor (NGI, Copley, Nottingham, UK). A thin layer of silicon grease (Slipicone; DC Products, Waverley, VIC, Australia) was coated onto the impactor stages to minimize particle bounce prior to the test. Approximately 12.3 mg of spray-dried RDV−SA cocrystal powders (equivalent to 10 mg of RDV) were loaded into a size 3 hydroxypropyl methylcellulose capsule (Capsugel, West Ryde, NSW, Australia), which were aerosolized by BreezhalerX (Novartis Pharmaceuticals, Hong Kong, China) at a flow rate of 60 L/min for 4 s. Known amounts of EtOH were used for rinsing RDV and SA in all stages. The solutions were subsequently filtered by 0.45 μm nylon syringe filters and assayed by HPLC. To ensure RDV and SA are not absorbed by the nylon filters, drug solutions at known concentrations were filtered, followed by measurement of their concentrations before and after filtration by HPLC analysis. No significant differences in RDV and SA concentrations were observed (P>0.05). The recovered dose, emitted fraction (EF), fine particle fraction (FPF), mass median aerodynamic diameter (MMAD), and geometric standard deviation (GSD) were calculated according to an established method(Weng et al., 2019). The EF referred to the fraction of powder that exited the inhaler to the total recovered dose. The FPF was the mass fraction of the particles <5 μm with respect to the recovered dose. The recovered dose was defined as the sum of powder mass assayed on all the parts.

### 6.9 Measurement of Content Uniformity

The quality of the RDV−SA DPI formulation was examined according to a modified protocol reported by Flament et al. (Flament et al., 2004) The quantity of RDV in aliquots (12.3 mg) of sampled RDV−SA powder, i.e., the amount of powder in each capsule tested in the NGI experiment, was analyzed. Each aliquot of sample was placed in a 10 mL volumetric flask and made up to the volume with methanol. Three aliquots were withdrawn from each sample, followed by HPLC analysis. From the triplicated results of RDV content in the samples, the average RDV content was calculated, and the content uniformity of the formulation was assessed by using the coefficient of variation (COV).

### 6.10 Scanning Electron Microscopy (SEM)

The particle morphology of the spray-dried RDV cocrystal was observed by field emission scanning electron microscopy (Hitachi S-4800 FEG, Hitachi, Tokyo, Japan). The powders were sprinkled onto carbon adhesive tape mounted on SEM stubs. Any sample not adhering to the tape was removed by compressed air. A sputter coater (Bal-tec SCD 005 Sputter Coater, Bal-Tec GmbH, Schalksmühle, Germany) was used to coat the powder with approximately 11 nm gold-palladium alloy in two cycles (60 s each) to create a conductive layer and avoid overheating.

### 6.11 In-Vitro Drug Release Study

To study the drug release profile of the cocrystal systems, equimolar amount (0.017 mol) of RDV (10 mg), RDV−SA (12.3 mg), and physical mixture of RDV (10 mg) and SA (2.3 mg) powders were separately poured into jacketed beakers containing 20 mL pH 7.4 simulated lung fluid 3 (SLF3)(Marques et al., 2011). All powders were sifted with a diameter under 63 μm to control the particle size variation. The solution was stirred at 75 rpm on a magnetic stirrer for a period of 120 min at 37±0.5° C. At designated time points of 2.5, 5, 7.5, 10, 15, 20, 30, 45, 60, 90, and 120 min, 500 μL of the dissolution medium was withdrawn and replaced with an equal volume of fresh medium. The sample solution was filtered through 0.45 μm nylon syringe filters and assayed by HPLC.

### 6.12 pH Determination

Equimolar amount (0.017 mol) of RDV (10 mg), SA (2.3 mg), and RDV−SA (12.3 mg) powders were dispersed into 20 mL pH 7.4 SLF3(Marques et al., 2011), respectively. The solution containing the samples was stirred at 75 rpm with a magnetic bar at 37° C., followed by in situ pH measurement at designated time points, i.e., 2 s, 5 s, 10 s, 15 s, 30 s, 45 s, 1 min, 1.5 min, 2 min, 3 min, 5 min, and 10 min, using a pH meter (Lab 850, Schott Instruments, United Kingdom).

### 6.13 MTT Cell Viability Assay

A549 cells were cultured in DMEM/F12 medium supplemented with 10% (v/v) FBS and 1% (v/v) antibiotic-antimycotic at 37° C. in a humidified incubator with 5% CO₂. On the day prior to treatment addition, approximately 2×10⁴ cells/well were seeded into 96-well plates and incubated overnight. After full attachment, the cells were treated for 24 hours with raw RDV, raw SA, physical mixture of RDV and SA, or the optimized spray-dried RDV−SA formulation (SD2) at concentrations of 0.05-10 μM, obtained by dissolving treatments in DMSO and subsequent dilution with complete DMEM/F12 medium. The highest concentration (10 μM) was selected based on a clinical study which evaluated the safety and pharmacokinetics of intravenous doses of RDV in healthy subjects(Humeniuk et al., 2020), where the highest plasma concentration of RDV observed was 4420 ng/ml (˜7.8 μM). Upon the treatment, the cells were incubated with 200 μL of MTT reagent (0.8 mg/mL) for 3 hours at 37° C. 100 μL of isopropyl alcohol was then added to each well to solubilize the formazan dye crystals and the absorbance at 570 nm was measured using a microplate spectrophotometer (Thermo Scientific™ Multiskan™ GO, Thermo-Fisher, Waltham, MA, USA). Cell viability (%) was calculated by dividing the absorbance of treated cells by the absorbance of cells from the control group with equivalent concentration of DMSO as the treatment group. Six replicates were made for each concentration and the whole MTT assay was repeated in triplicate.

### 6.14 Solubility Measurement

The solubilities of RDV and SA in different organic solvents (EtOH, MeOH, ACE, ACN, and CHF) were determined by adding excess solid in screw capped test tubes with 2 mL of organic solvent and shaking for 72 h. Samples were filtered through 0.45 μm membrane filters, followed by dilution to appropriate concentrations for the HPLC assay.

### 6.15 Statistical Analysis

Statistical analysis was performed by one-way ANOVA using PRISM 7 software (Graphpad Software Inc., San Diego, CA, USA). Differences were considered as statistically significant if P<0.05.

### 7. RESULTS AND DISCUSSION

### 7.1 Cocrystallization of Remdesivir with Benzoic Acid Derivatives

Attempts were made to cocrystallize RDV with BA, SA, ASA, and GA using LAG in five organic solvents (EtOH, MeOH, ACE, ACN, and CHF). Solvents were selected to represent different polarities and relative solubilities of the cocrystal formers.

RDV polymorphic form II was selected as a representative polymorph. Form II is the most thermodynamically stable RDV polymorph exhibiting a melting point of 138° C., which is 27° C. higher than form I (111° C.), but with a slower dissolution. The higher stability of RDV form II is associated with the multiple zigzag-shaped energy frameworks that crosslink to construct an overall 2D grid via stronger intermolecular interactions. In some embodiments, RDV forms I, III or IV can also be used to form a SA cocrystal according to the present disclosure.

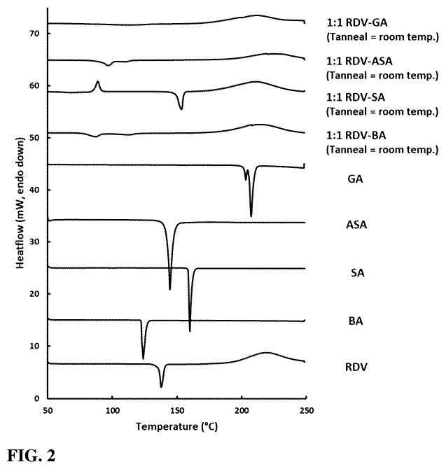

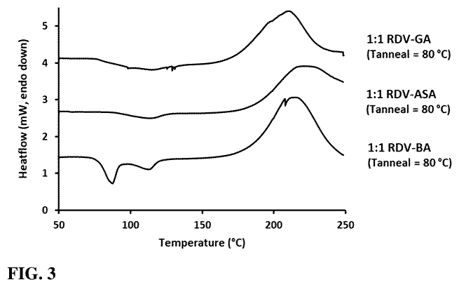

Only a phase pure 1:1 RDV−SA cocrystal was successfully obtained, whereas samples prepared with other three coformers led to high degree of amorphization despite the efforts in adjusting the processing and annealing parameters (FIGS. 2 and 3). DSC traces in FIG. 4A showed that RDV−SA produced by LAG in different solvents displayed a melting endotherm at 152° C. to 154° C., which lies between those of RDV (137.82° C.) and SA (159.79° C.).

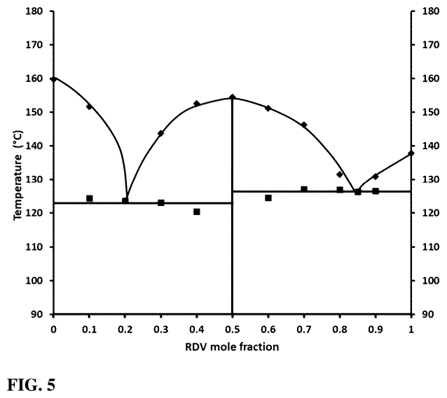

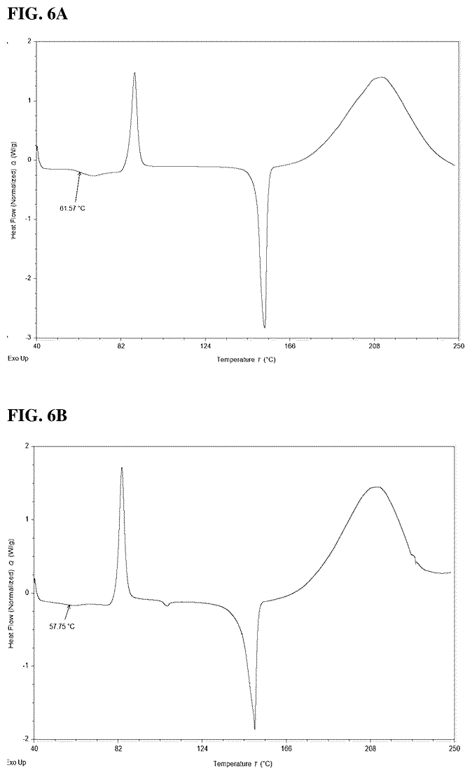

To further confirm the new phase, a temperature-composition phase diagram was constructed using binary mixtures of RDV and SA through DSC analysis (FIG. 5). Binary phase diagram construction via DSC analysis has been commonly applied in characterizing solid solution and cutectic mixture for a given system. It has been adopted for conducting efficient and comprehensive cocrystal screening. The phase diagram of RDA-SA revealed a local maximum melting temperature at 0.5 RDV mole fraction, confirming its 1:1 stoichiometry. Two eutectic points were located at 0.2 and 0.85 RDV mole fractions with eutectic melting at 122.89° C. and 126.34° C., respectively. Notably, in LAG samples using EtOH and ACN, the cocrystal melting was accompanied by an exothermic peak at around 80° C., which was attributed to recrystallization of the remnant amorphous material as a glass transition was observed at ˜61° C. (ACN) and ˜58° C. (EtOH), leading to fluctuation in baseline (FIG. 6). The molecular diffusion process involved in cocrystallization via mechanochemistry is known to be mediated by one of three high energy transition intermediates with enhanced molecular mobility (FrišČiĆ and Jones, 2011), including (i) vapor, (ii) liquid eutectic, and (iii) amorphous phases. Formation of an amorphous intermediate is regarded as the most common mechanism especially when limited mass transfer happens (Friscic and Jones, 2009). Hence, solid-state grinding resulted in partial cocrystallization between RDV and SA, whereas the rest systems remained as amorphous mixture.

Next, the LAG samples were subject to successive annealing treatment at 80° C. for 30 minutes. FIG. 4B illustrated that all thermally annealed samples exhibited a sharp endotherm at around 153° C., while the recrystallization peak disappeared. The cocrystal melting was found at a higher temperature (p=0.0027) and molar enthalpy of fusion (ΔH_{f}, p=0.0002) compared with non-thermally annealed RDV−SA (Table 2). This could be ascribed to the achievement of higher crystallinity and packing efficiency along annealing, which led to stronger intermolecular interactions in RDV−SA crystal lattice. The vast majority of organic cocrystals reported in literature possessed ΔH_{f} above their constituted counterparts (Chow et al., 2012; Cui et al., 2019; Good and Rodriguez-Hornedo, 2009; Surov et al., 2014; Wong et al., 2018). Yet, what have been observed in these thermally activated species was a consequence of reduction in crystal lattice strength upon cocrystallization, i.e., with a ΔH_{f} either lying between or below their individual constituents (RDV: 55.44 KJ/mol, SA: 24.85 KJ/mol, thermally annealed RDV−SA: 50.29 KJ/mol, non-thermally annealed RDV−SA: 42.81 KJ/mol) (Wong et al., 2022a). This phenomenon is consistent with a previous report that the ΔH_{f} of thermally activated dexamethasone-catechol and dexamethasone-resorcinol cocrystals were also around 60-70% lower than the sum of those of their individual constituents (Wong et al., 2022a). Further investigation with a larger sample size of this cocrystal category is warranted to elucidate such correlation between their supramolecular chemistry, thermal properties, and cocrystallization efficiency. The key thermal properties of the RDV−SA cocrystal system are tabulated in Table 2.

TABLE 2
Melting temperature and heat of fusion of RDV, SA, and RDV-SA cocrystals produced
by liquid-assistend grinding (LAG), and spray drying (SD) in ACN (n = 3).

| | Before Thermal | | After Thermal | | | |
|---|---|---|---|---|---|---|
| | Annealing | | Annealing | | | |
| | (Tₐₙₙₑₐₗ = room temp.) | | (Tₐₙₙₑₐₗ = 80° C.) | | p value | |
| | Melting | | Melting | | Melting | |
| | Temperature | ΔH_{f} | Temperature | ΔH_{f} | Temp | ΔH_{f} |
| | (° C.) | (kJ/mol) | (° C.) | (kJ/mol) | (° C.) | (kJ/mol) |
| RDV | 137.82 ± 0.3 | 55.44 ± 1.2 | N/A | | N/A | |
| SA | 159.79 ± 0.5 | 24.85 ± 0.7 | N/A | | N/A | |
| LAG_RDV- | 151.84 ± 0.3 | 42.81 ± 0.5 | 153.36 ± 0.2 | 50.29 ± 0.8 | 0.0027 | 0.0002 |
| SA (ACN) | | | | | | |
| 0.SD_RDV- | 151.01 ± 1.5 | 44.12 ± 0.9 | 154.24 ± 1.1 | 58.22 ± 0.6 | 0.0398 | 0.00004 |
| SA (ACN) | | | | | | |

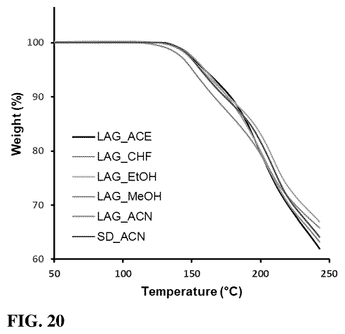

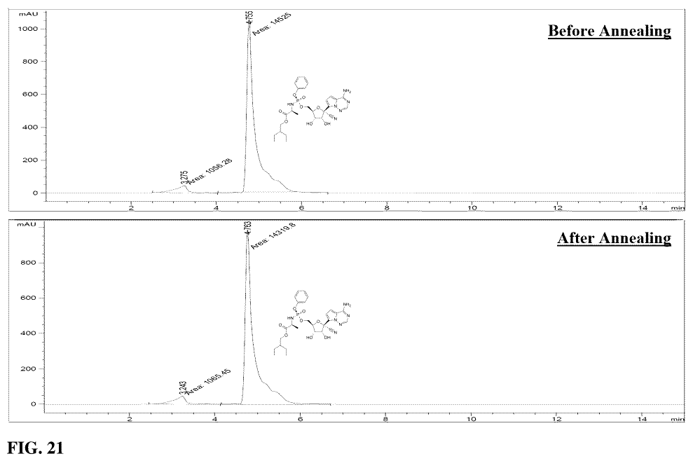

The TGA data indicated the LAG RDV−SA produced using different organic solvents (EtOH, MeOH, ACE, ACN, and CHF) are with insignificant weight loss prior to the melting, suggesting that negligible solvent was concealed in the samples (FIG. 20). The tiny amount of solvent added during LAG is expected to have solely a catalytic role in facilitating the formation of RDV cocrystal. Besides, considering that thermal treatment may affect parts of RDV via interference of the amide and the ester groups (Hamdy et al., 2021), the thermal stability of RDV−SA was assessed by measuring the RDV contents before and after thermal annealing in triplicate via HPLC. Table 3 illustrates that the RDV assay in the LAG RDV−SA cocrystal did not change significantly compared to the raw RDV upon a 30-min annealing treatment at 80° C. (i.e., RDV−SA cocrystal: 98.91±1.25%; raw RDV: 99.28±0.27; p=0.12). In addition, HPLC chromatograms revealed that no shift of RDV peaks and no additional degradation products were observed after thermal annealing (FIG. 21). This indicated that RDV in the RDV−SA cocrystal was chemically stable during the annealing process. Based on the results, the risk of RDV degradation can be minimized by controlling the annealing time to the shortest possible, preferably 30 minutes.

TABLE 3
Thermal stability of RDV cocrystals under annealing at 80° C.

| | Pure RDV | RDV-SA_LAG |
|---|---|---|
| Annealing temperature | 80° C. | |
| Annealing time | 30 min | |
| % Assay of RDV after exposed to | 99.28 ± 0.27 | 98.91 ± 1.25 |
| thermal annealing (%) | | |

| | |
|---|---|
| p value | 0.12 |

PXRD analysis provides deeper insights into delineating the thermally activated cocrystallization process of the RDV−SA system. As shows in FIG. 7A, after undergoing thermal annealing at 80° C. for 30 minutes, RDV−SA prepared by LAG in different organic solvents displayed essentially the same PXRD patterns, where a series of distinct diffraction peaks are discernible (2θ=5.14°, 5.62°, 8.32°, 15.00°, 16.52°, 18.58°, and 21.48°, Table 4).

TABLE 4
PXRD data below 30 degree two-theta of the thermally
annealed LAG RDV-SA cocrystal powder.

| 2-theta | d | Height | FWHM |
|---|---|---|---|
| (degree) | (Angstrom) | (normalized) | (degree) |
| 5.1573 | 17.120882 | 100 | 0.1692 |
| 8.3428 | 10.589426 | 38.17 | 0.1684 |
| 9.7993 | 9.018522 | 13.67 | 0.176 |
| 10.3529 | 8.537464 | 3.95 | 0.1643 |
| 10.9276 | 8.089771 | 2.9 | 0.1506 |
| 11.2877 | 7.832448 | 14.89 | 0.1411 |
| 12.5988 | 7.020175 | 3.99 | 0.1755 |
| 13.048 | 6.779501 | 13.43 | 0.1489 |
| 13.6568 | 6.478571 | 2.5 | 0.1008 |
| 14.52 | 6.095331 | 23.45 | 0.9021 |
| 14.752 | 5.99999 | 20.65 | 0.9021 |
| 14.992 | 5.904475 | 89.1 | 0.9021 |
| 16.0117 | 5.530686 | 9.8 | 0.0428 |
| 16.5211 | 5.361268 | 62.37 | 0.0434 |
| 16.6949 | 5.30586 | 37.87 | 0.0436 |
| 16.9401 | 5.229593 | 13.19 | 0.0439 |
| 17.4203 | 5.086504 | 1.64 | 0.0445 |
| 18.3138 | 4.840299 | 38.06 | 0.1206 |
| 18.5673 | 4.774779 | 88.87 | 0.1214 |
| 19.5521 | 4.536461 | 18.78 | 0.1249 |
| 19.7108 | 4.500288 | 11.99 | 0.1254 |
| 20.304 | 4.370131 | 3.64 | 0.3297 |
| 20.536 | 4.32128 | 1.47 | 0.3297 |
| 21.112 | 4.204666 | 6.59 | 0.3297 |
| 21.504 | 4.128896 | 39.18 | 0.3297 |
| 22.192 | 4.002421 | 10.99 | 0.3297 |
| 23.168 | 3.835969 | 71.14 | 0.0395 |
| 23.5832 | 3.769366 | 12.23 | 0.0398 |
| 24.0882 | 3.691476 | 19.73 | 0.0401 |
| 24.3081 | 3.658573 | 6.15 | 0.0403 |
| 24.7334 | 3.596622 | 7.04 | 0.0406 |
| 25.3602 | 3.509132 | 26.84 | 0.041 |
| 25.7036 | 3.463027 | 13.65 | 0.0413 |
| 26.4402 | 3.368187 | 25.54 | 0.0418 |
| 26.7198 | 3.333577 | 15.37 | 0.042 |
| 27.4243 | 3.249522 | 11.88 | 0.0425 |
| 28.7268 | 3.105076 | 6.1 | 0.1459 |
| 29.4963 | 3.025798 | 15.67 | 0.1478 |

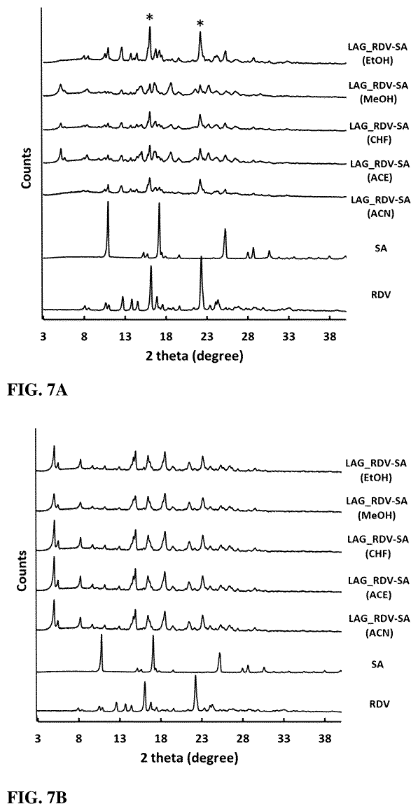

The major characteristic peaks corresponding to the parent constituents were absent (RDV: 2θ=16.15°, 22.29°, SA: 2θ=10.9°, and 17.16°). Since the new peaks found at higher angles (e.g., 2θ=15.00°, 18.58°) do not exist in the PXRD patterns of other metastable RDV polymorphs, one can exclude the possibility that the change of PXRD patterns was due to polymorphic transformation of RDV. In contrast, all LAG samples retained the characteristic peaks of RDV when annealing occurred at ambient conditions (as denoted by *), albeit some signature peaks of the RDV−SA cocrystal were observed in a few batches of samples (FIG. 7A). It is worth noting that the PXRD peaks in the non-thermally activated LAG samples had relatively weak intensities, suggesting the existence of partial amorphous content generated during the grinding. Annealing at a temperature higher than the glass transition temperature (T_{g}=˜57-61° C., FIG. 6) resulted in recrystallization of the remnant amorphous content into the RDV−SA cocrystal. This again confirmed that the success of RDV−SA cocrystallization was kinetically driven by two major steps, viz. (i) sufficient mechanical force to fracture the crystal structure of cocrystal formers into a unstable amorphous intermediate; and (ii) thermal activation to facilitate structure relaxation resulting in the crystallization of RDV−SA (Jayasankar et al., 2006; Oguchi et al., 2002). Thermal annealing at high temperature only and without co-grinding of the physical mixtures of RDV and SA failed to produce phase pure RDV−SA cocrystals (FIG. 8), indicating that both (i) and (ii) steps are essential to the RDV−SA cocrystallization.

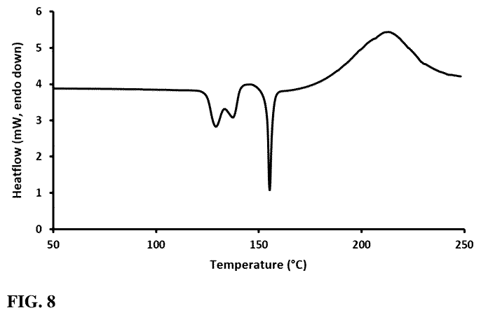

To define the optimal LAG conditions for RDV−SA cocrystal formation, the effects of annealing temperature and annealing time on the transformation of RDV−SA from amorphous to cocrystal in solid state was further investigated. The DSC traces in FIG. 9 indicated that upon brief exposure of the grounded sample to elevated temperature at 80° C. (>T_{g}) for 30 min, the recrystallization peak associated with the amorphous content gradually disappeared. The transformation was possible by thermally aided strengthening of hydrogen-bonding interactions in the amorphous intermediate phase. When a lower annealing temperature of 60° C. was employed, the recrystallization of RDV−SA appeared to be retarded since the materials are more viscous with lower molecular mobility in the brittle glassy state, compared with its supercooled liquid state counterpart. Hence, short-range disordered coamorphous solid in high-energy states was favored. The glass transition was noticeable until 30 minutes of thermal annealing, where the T_{g} of the amorphous phase decreased when the annealing time increased. This observation supports the view that an annealing temperature higher than the T_{g} is required to drive an efficient recrystallization of RDV−SA from the amorphous intermediate.

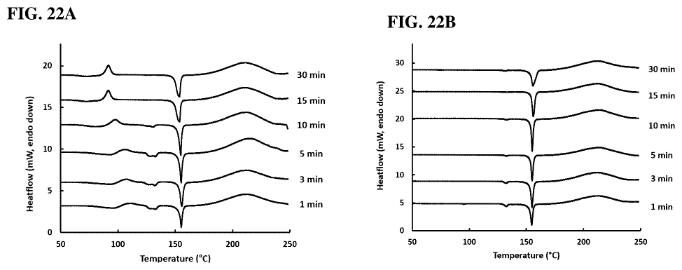

The purities of the RDV−SA cocrystals obtained at different grinding times (1 min, 3 min, 5 min, 10 min, 15 min, and 30 min) were also evaluated by DSC. FIG. 22A shows that co-grinding of RDV with SA at ambient condition initially resulted in a hybrid phase comprising mainly a physical mixture of individual parent constituents and a small fraction of RDV−SA amorphous intermediate. With an increase in grinding time, the broad endothermic peak at around 130° C. which represents the unreacted coformers gradually shrank while the glass transition shifted to a lower temperature, accompanied by a more intense exothermic peak formed at around 80° C. due to recrystallization. No further significant change was observed when the grinding time was increased from 15 min to 30 min. Each RDV−SA sample formed at specific times during the grinding process was then thermally annealed at 80° C. The DSC traces again indicated a grinding time-dependent RDV−SA cocrystal formation under thermal stress. Similar effect was also demonstrated in a previous report regarding the indomethacin-nicotinamide cocrystal formation (Lin et al., 2014). FIG. 22B showed that albeit small, the endothermic peak at around 130° C. was still consistently found in the RDV−SA samples unless the grinding time was increased to 15 min. Considering that the quantity of amorphous phase appeared to increase on extended grinding, a 15-min grinding was deemed as the optimal shortest grinding time for obtaining a pure RDV−SA cocrystal while maintaining a good crystallinity.

Although successful cases in annealing assisted mechanochemical syntheses of pharmaceutical cocrystal at high temperature are limited, it is deemed a valuable technique in metal complex chemistry (Kuroda et al., 2009). Solid heating of metal complexes after mechanochemical treatments can activate individual molecules by promoting the dissociation of weakly coordinated small ligands, e.g., N₂, NH₃, and H₂O, thus generating vacant coordination sites for accepting additional coordination (Bianchini et al., 1991; Tsuchiya et al., 1974). Preparation of new crystalline salt which comprised protonated ligands and a chloro complex was also reported using the said method (Adams et al., 2005; Minguez Espallargas et al., 2006). These studies suggested the significance of thermal annealing lies in promoting diffusion and rearrangement of molecules, and sometimes chirality transfer in solid state to form new crystal phases (Kuroda et al., 2009). The thermally activated “on-off” control on phase transformation to a pharmaceutical cocrystal is not trivial. The utility of cocrystals in medicine has been restricted by erratic cocrystal dissociation, hindering their clinical translation (Wong et al., 2021). Unexpected cocrystal dissociation into individual constituents during long-term storage, especially under high humidity condition, can introduce quality issues and eliminate the beneficial properties brought by cocrystallization. The thermal activation functionality of the RDV−SA cocrystal thus may offer an intriguing opportunity to minimize the risk of cocrystal dissociation as the cocrystal can be activated only prior to the uses.

Apart from the thermal effect, the nature of organic solvents in LAG appeared to dictate the purity of RDV−SA cocrystal. The signature PXRD diffraction peaks of RDV−SA cocrystal (i.e., 2θ=5.14°, 5.62°, 15.00°, and 18.58°) could only be seen in samples using MeOH, CHF and ACE prior to thermal annealing, but not EtOH and ACN (FIG. 7A). This echoes the DSC data where recrystallization exotherms were found in samples using EtOH and ACN. These observations can be explained by a larger solubility difference between RDV and SA in EtOH and ACN. The important role of the relative solubilities of cocrystal formers in solution-based cocrystallization which is mediated by a minimal amount of solvent was exemplified in a theophylline/caffeine-tartaric acid cocrystal system (Friščić et al., 2009). The solubility ratios of SA to RDV in different solvents were ranked in the following order: ACN>CHF>EtOH>MeOH>ACE (Table 5).

TABLE 5
Equilibrium solubilities of RDV and
SA in different solvents at 25° C.

| | Solubility (mg/mL) | | Solubility Ratio |
|---|---|---|---|
| Solvent | RDV | SA | (SA/RDV) |
| Ethanol | 34.19 ± 2.29 | 279.69 ± 11.31 | 8.18 |
| Methanol | 191.05 ± 13.34 | 333.40 ± 24.76 | 1.745 |
| Acetone | 66.40 ± 5.34 | 28.34 ± 0.45 | 0.43 |
| Acetonitrile | 5.68 ± 0.60 | 289.08 ± 8.45 | 50.89 |
| Chloroform | 3.86 ± 0.30 | 75.18 ± 10.02 | 19.48 |

The markedly higher solubility ratio in ACN (50.89) than in other solvents suggests a much stronger propensity of the precipitation of the less soluble component (RDV) due to their incongruent solubility. Cocrystallization outcome is associated with the saturation levels achieved in the reaction, i.e., formation of cocrystal is favorable when both cocrystal formers remain saturated in the solvent (Friščić et al., 2009). However, in incongruent systems of RDV−SA in ACN, following the formation of an amorphous intermediate during LAG, the highly soluble SA induced dissociation of the amorphous to the individual constituents, while the concentration of RDV rapidly reaches the saturation point, causing precipitation (Friščić et al., 2009). On the other hand, although solubility ratio of coformers plays a role in influencing cocrystal phase purity, it is not necessarily the only contributing factor involved, especially for kinetic-driven cocrystal systems. The solubility ratio of SA to RDV is around 2.5 times higher in CHF (19.48) than in EtOH (8.18), whereas the purity of the resulting cocrystal appeared to be higher when CHF, the only non-polar solvent, was used. It is surprising that CHF with a higher solubility ratio could afford a relatively more efficient RDV−SA cocrystallization as compared with EtOH. Such a selectivity may be attributed to other solvent-dependent molecular assembly and structure-directing effects associated with the different polarities between CHF and EtOH, which regulate the nucleation and metastable zone width of the solid forms. Polar solvents, such as EtOH, can have a propensity to interact competitively with the hydrogen-bond donating sites of the coformers. Blockage of the binding sites therefore renders the cocrystallization reaction less favorable (Lombard et al., 2020; Sarmah et al., 2019). On the contrary, the non-polar solvent CHF plays a minimal role in preventing direct contact between cocrystal formers due to the absence of hydrogen bonding donor/acceptor sites. In addition, the polarity of solvents can profoundly influence the crystal packing arrangement. For example, with an increased solvent polarity, the tautomeric equilibrium of curcumin in solution can shift from the diketone to the enol structure, which favored the formation of curcumin-phloroglucinol cocrystal. Similarly, the use of non-polar CHF could interfere with dimerization during 2,6-dihydroxybenzoic acid polymorph formation, and abolished the “open” conformational polymorph of a pyrazolyl urea ligand during crystallization, compared with other polar solvents.

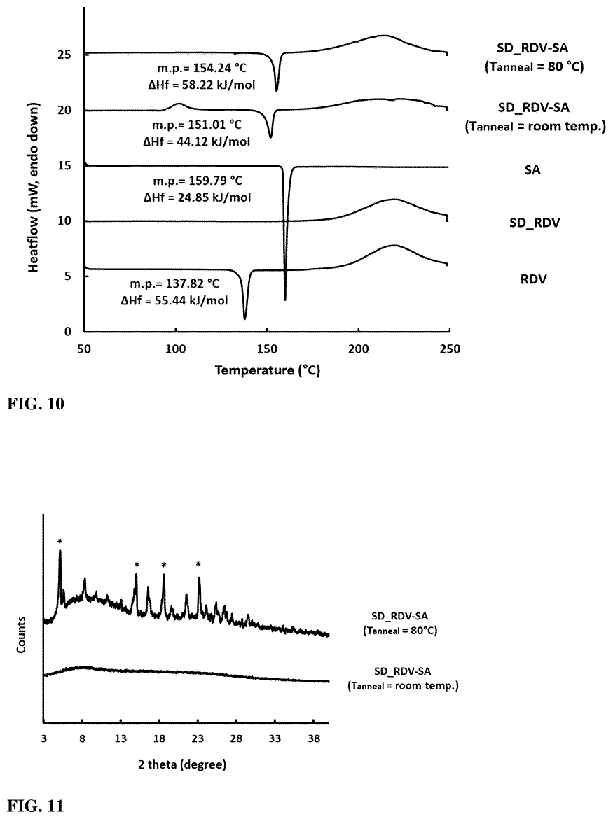

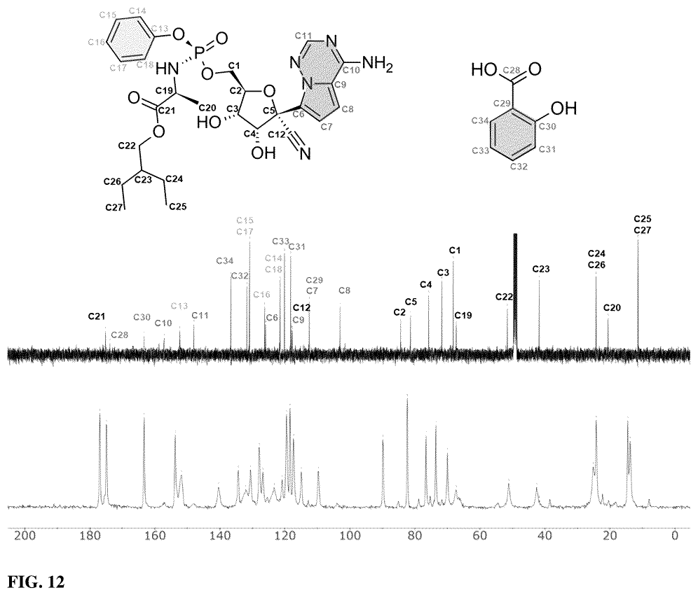

However, it should be noted that such solvent effect was negated after giving sufficient time for thermal annealing, as confirmed by the essentially the same PXRD patterns of different RDV−SA samples prepared using EtOH, McOH, ACE, ACN, and CHF (FIG. 7B). In some embodiments, the present disclosure incorporates thermal annealing as a practical strategy in cocrystal screening methodology. Since there is no discrepancy observed in the LAG samples produced from different solvent systems after thermal annealing, ACN with the highest solubility ratio, was used to reproduce RDV−SA cocrystal using spray drying, for the purpose of inhalable dry powder formulation development (FIG. 10 and FIG. 11). Selection of ACN as the model solvent would better observe difference in cocrystallization caused by the thermally activation process. Initial trials of unit cell indexing from the PXRD profile of the thermally annealed LAG RDV−SA cocrystal were in vain, as indicated by a large number of possible sets of unit cell parameters. After neglecting the non-obvious peaks that originated from the individual starting materials RDV and SA in the cocrystal diffraction profile, a unit cell with reasonable dimensions were indexed successfully. Additional insights into the unit cell determination process and information of the chemical environments of RDV and SA molecules in the crystal lattice were supported by the Proton-decoupled ¹³C CP-MAS solid-state NMR spectrum of the cocrystal. With the comparison to the corresponding NMR signals in solution, solid-state resonance peaks from carbon atoms of both RDV and SA in the cocrystal were unambiguously assigned (FIG. 12). The narrow resonance peaks in the spectrum corroborated that the RDV and SA molecules were well-ordered, and the sample is highly crystalline. Investigation of the number of isotropic resonance lines in the spectrum reveals the presence of a single set of resonance peaks from RDV and SA molecules, which indicated the asymmetric unit of the thermally annealed LAG RDV−SA cocrystal comprises one pair of crystallographically independent RDV and SA molecules (Z′=1). This finding implies the asymmetric unit contains 52 non-hydrogen atoms (42 from RDV and 10 from SA), and it is consistent with the unit cell parameters achieved from indexing the diffraction pattern with the unit cell volume (1826.53(17) Å³) and the non-centrosymmetric monoclinic space group P2₁ assignment. Simulated annealing followed by Rietveld refinement of the diffraction pattern revealed the cocrystal structure. Selected crystallographic data and refinement details of the cocrystal are provided in Table 6.

TABLE 6
Selected crystallographic data and structure refinement
results of thermally annealed LAG RDV-SA cocrystal.

| | |
|---|---|
| Moiety formula | C₂₇H₃₅N₆O₈P, C₇H₆O₃ |
| Formula weight | 740.70 |
| Crystal system | Monoclinic |
| Space group | P2₁ |
| Temperature/K | 293 |
| Appearance | White powder |
| a/Å | 18.2598(10) |
| b/Å | 5.5887(2) |
| c/Å | 19.3032(11) |
| α/° | 90 |
| β/° | 111.991(4) |
| γ/° | 90 |
| Volume/Å³ | 1826.53(17) |
| Z | 2 |
| ρ_{calc}/gcm³ | 1.347 |
| 2θₘᵢₙ, 2θₘₐₓ, 2θₛₜₑₚ/° | 3.000/59.976/0.008 |
| N_{reflection}, N_{data}, Nₚₐᵣₐₘₑₜₑᵣ | 639/7123/195 |
| Final Rₚ, R_{wp}, Rₑₓₚ, | 0.044/0.061/0.013/0.114 |
| R_{Bragg} | |

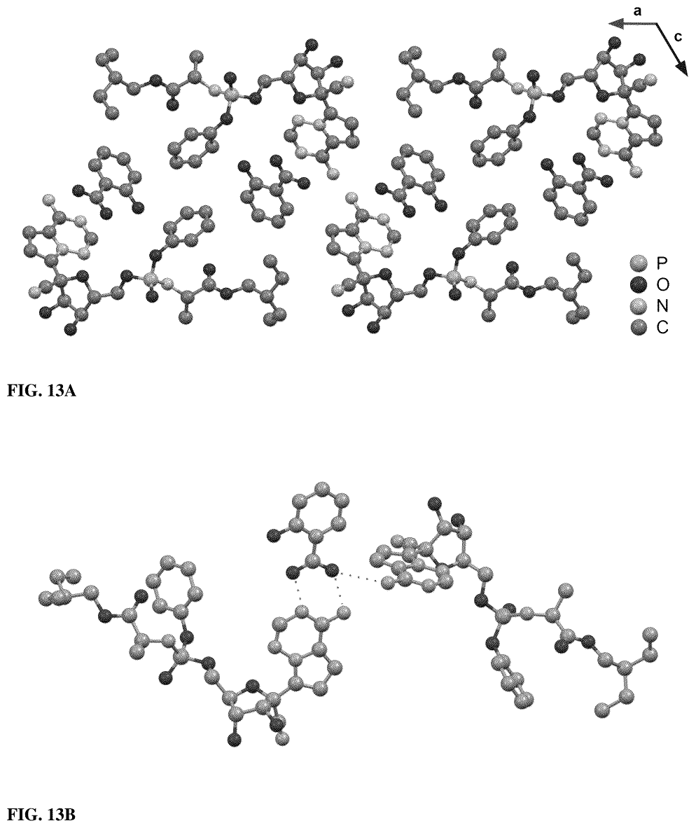

In the refined model, intermolecular interactions are observed between the 4-aminopyrrolotriazine moiety of RDV and the carboxylic acid group of SA. The 4-aminopyrrolotriazine-carboxylic acid two-point recognition heterosynthon resembles the well-studied 2-aminopyridine-carboxylic acid supramolecular heterosynthon motif. The distances between the hydrogen-bonded atom pairs 2.83 (2) Å for O9—H . . . N3 and 2.93 (3) Å for O10 . . . H—N4 are in the typical intermolecular hydrogen bonding distance range (Bis and Zaworotko, 2005). Due to the restricted rotational motion of the C—C single bonds in the 2-ethylbutoxy group within the crystal lattice, the two terminal CH₃ groups in the RDV experience different surrounding environments. C25 is in close proximity to the ester oxygen, methyl carbon of the alanine moiety, and methanetriyl and methylene carbons of the 2-ethylbutoxy chains of adjacent RDV molecules, whereas C27 is neighboring to the aromatic ring systems of the phenolic ring in the same molecule and the adjacent SA molecule. The different chemical environments experienced by the two terminal CH₃ groups are consistent with the solid-state NMR spectrum, as two resonance peaks (11.6 ppm and 12.4 ppm) were observed. The three-dimensional crystal structure of the cocrystal and the intermolecular interactions within the cocrystal are depicted in FIG. 13.

FTIR analysis features the intermolecular interactions engaged in the thermal activation cocrystallization of RDV−SA (FIG. 14). The FTIR spectra of pure RDV and the non-thermally annealed RDV−SA shared a large degree of similarity in major absorption peaks (Table 7), despite subtle differences in the peak shape corresponding to N—H and C═O stretching. A complex band region at 3320-3427 cm⁻¹ was assigned to the superposition of N—H and O—H stretching. In contrast, spectral peak shifts appeared for various functional groups in the thermally annealed RDV−SA cocrystal compared with pure RDV, implying a change in chemical environment upon cocrystallization. For example, the peak reflecting N—H stretching dramatically shifted from 3427 cm⁻¹ to 3468 cm⁻¹. The prominent peak originally located at 3171 cm⁻¹ in pure RDV diminished, replaced by a broad absorption peak ranged from 3100 to 3300 cm⁻¹, corresponding to phenolic O—H stretching. Notably, the C—N stretching shifted from 1021 cm⁻¹ to a higher frequency, i.e., 1042 cm⁻¹. This implies their active role in engaging in the intermolecular interactions, e.g., hydrogen bonding between the 4-aminopyrrolotriazine moiety of RDV and the carboxylic acid group of SA, in cocrystal formation.

TABLE 7
Key features in the FTIR spectra of RDV, SA,
RDV-SA annealed at different temperatures.

| | N—H | O—H | C═O | C—N |
|---|---|---|---|---|
| | stretching/ | stretching/ | stretching/ | stretching/ |
| Sample | cm⁻¹ | cm⁻¹ | cm⁻¹ | cm⁻¹ |
| RDV | 3424 | 3171 | 1723, 1660 | 1246, 1042 |
| SA | — | 3420, 3239 | 1656, 1656 | — |
| RDV-SA | 3427 | 3174 | 1724, 1662 | 1248, 1042 |
| (Tₐₙₙₑₐₗ = room | | | | |
| temp.) | | | | |
| RDV-SA | 3468 | 3100-3300 | 1742, 1673 | 1259, 1021 |
| (Tₐₙₙₑₐₗ = 80° C.) | | (broad) | | |

### 7.2 Inhalable RDV−SA Development and Aerosol Performance

Different formulation strategies can be used to produce inhaled RDV powders. For example, thin film freezing can be used to develop an inhaled RDV DPI formulation with 93.0% FPF_{<5 μm} and 0.82 μm MMAD. Inhaled RDV liposomes with satisfactory aerosol performance can also be used. According to the present disclosure, spray drying was employed since it is a proven technique for fabricating inhalable cocrystal DPIs with suitable aerosol performance for deep lung delivery (Alhalaweh et al., 2013; Weng et al., 2019; Wong et al., 2022b). Its attractiveness lies in the precise control of particle properties in a single continuous step, including aerodynamic particle size, dispersibility, and morphology, etc. (Hadiwinoto et al., 2018; Ohtake et al., 2020)

As contemplated herein, RDV−SA cocrystal was successfully reproduced using spray drying in ACN followed by thermal annealing at 80° C., of which the DSC trace was consistent with that produced by LAG (FIG. 10). No crystallization was observed upon heating. In contrast, spray drying of pure RDV resulted in fully amorphous powders, showing a glass transition at around 60° C. in the DSC thermogram (FIG. 6C). Although the PXRD pattern of spray-dried RDV−SA formulation with thermal annealing exhibited relatively low crystallinity, the characteristic peaks of RDV−SA such as those located at 5.14°, 5.62°, 15.00°, and 18.58° remained to be observed (FIG. 11). Concerning the aerosol performance, formulation obtained from an initial attempt of spray drying (SD1) had a large MMAD (9.84±0.41 μm) and a low FPF (13.86±1.41% w/w) at an inspiratory flow rate of 60 L/min. The flow rate was set to 60 L/min as it can be achieved by most patients with chronic lung diseases (Altman et al., 2018; Liao et al., 2020; Wong et al., 2022b).

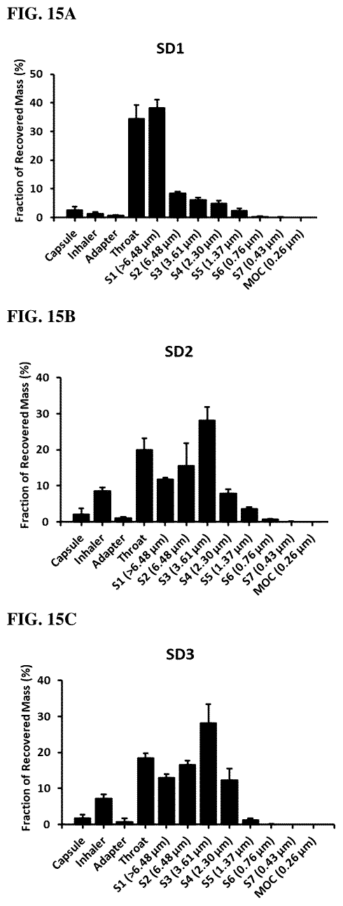

Clinical evidence demonstrated that patients suffering from multiple pulmonary comorbidities are at higher risk for severe illness from COVID-19. For example, the risk of contracting COVID-19 in patients with chronic obstructive pulmonary disease (COPD) is found to be 4-fold higher than in patients without COPD (Zhao et al., 2020). These comorbid patients with compromised lung function may not be able to generate sufficient inspiratory force (Mahler, 2017). Hence, it is clinically relevant to probe into whether a low inspiratory airflow, i.e., 60 L/min, could provide sufficient energy to disperse the powders. FIG. 15 indicated a large proportion of SD1 drug particles were trapped in the throat (34.55±4.79%). Only particles with aerodynamic diameters of 1-5 μm are considered as optimal for pulmonary drug delivery, further optimization on the spray drying process were performed.

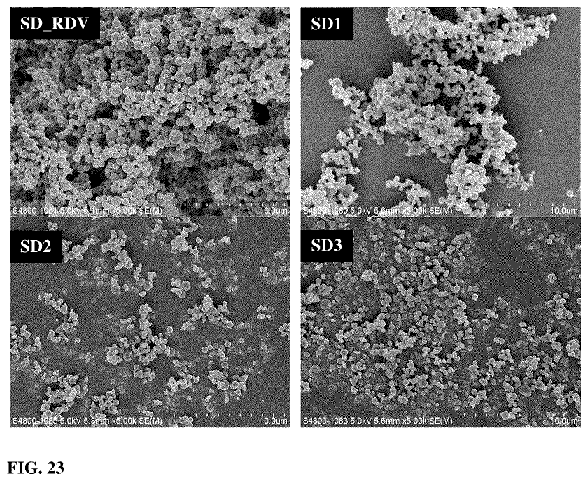

Our earlier study has applied a Quality-by-Design approach to systematically investigate the impacts of various critical processing parameters on the MMAD, FPF, and crystallinity of another antiviral inhalable cocrystal (Wong et al., 2022b). Prompted by the findings, the solute concentration and feed pump rate were reduced to produce SD2 and SD3 (Table 1). SD2 exhibited the most satisfactory MMAD (4.33±0.2 μm) and highest FPF (41.39±4.25% w/w), which is comparable to that of most commercial products. With a higher atomizing gas flow, the aerosol performance of SD3 was slightly inferior to that of SD2, yet still suitable for deep lung delivery. The NGI dispersion plot illustrates that after optimization, the powders deposited on stages 3-5 increased from 13.43% (SD1) to 39.88% (SD2), where the aerodynamic diameters fell within 3.61-0.76 μm (FIG. 15). The smaller MMAD of SD2 and SD3 compared with SD1 could be provoked by the reduced solute content in each atomized droplet and thus a longer interparticle distance with diffused nuclei was maintained. This also resulted in a decreased feed viscosity and minimized particle agglomeration, which is supposed to happen at a high degree of local supersaturation (Chen et al., 2020; Gonnissen et al., 2008; Hadiwinoto et al., 2018). In addition, the reduced feed pump rate was associated with a higher atomization energy, which promoted sufficient droplet fission to further reduce the size (Anandharamakrishnan, 2015; Santos et al., 2018). The atomizing gas flow alone was shown not to exert any statistically significant effect on the aerosol performance of an inhalable cocrystal powder (Wong et al., 2022b). However, in this study, a lower atomizing gas flow in SD2 was associated with a slightly smaller MMAD and higher FPF, when other processing parameters were identical. The particle morphology and surface property of the spray dried RDV−SA powders were further examined by scanning electron microscopy (SEM). The SEM images revealed that the morphology of SD1, SD2, and SD3 resembled that of the spray dried RDV, displaying a spherical-shaped structure with smooth surface (FIG. 23). The primary particle sizes of all formulations were similar. SD1, prepared at two times higher total solute concentration and feed pump rate, showed a high degree of agglomeration, whereas SD2 and SD3 appeared as discrete units. This confirmed that the superior acrosol performance of SD2 and SD3 was in fact due to their less aggregating structures, which contributed to higher FPFs and smaller MMADs during powder dispersion. Since the powders can casily undergo deagglomeration, it is anticipated that the specific surface area in contact with the lung fluid can be greatly enlarged and hence, leading to improved dissolution performance. It should be noted that the spray dried RDV−SA formulation was still partially amorphous after thermal annealing, which may be associated with undesirable physicochemical instability. Apart from manipulating the feed pump rate and the atomizing gas flow, it may be considered to mitigate amorphization by reducing the inlet air temperature, which was defined as a fixed parameter of 60° C. in the present study. For example, it has been reported that decreasing the inlet temperature by 40° C. resulted in an increase of particle crystallinity threefold from 22 to 72% during the spray drying of lactose solution (Das and Langrish, 2012). This could be ascribed to a longer particle drying time which allows the particles to rearrange themselves into a more crystalline form. A lower inlet temperature leads to greater moisture sorption in particles, where water acts as a plasticizer to enhance the rate of cocrystallization from the coamorphous phase (Jayasankar et al., 2006; Pekar et al., 2021). However, a decrease in feed temperature may increase feed viscosity causing an increase in droplet size produced in the atomizer (Chidavaenzi et al., 1997). This highlights the dilemma of manufacturing inhalable cocrystal dry powders exhibiting suitable particle sizes, without compromising the degree of crystallinity. To ensure RDV is distributed uniformly among the individual units, the average content and the content uniformity of RDV in the spray dried formulations (SD1, SD2, SD3) were also assessed. Table 8 showed that all formulations exhibited small variations in RDV content in different batches as reflected by the low coefficient of variations (COV≤5%), confirming that the uniformity of drug in cocrystal DPI is acceptable. Adopting different processing parameters of spray drying did not result in any significant difference in RDV contents (p>0.5).

TABLE 8
Average content and uniformity of drug content for the RDV-SA
dry powder formulations (COV = coefficient of variation).

| Formulations | RDV Content (mg) |
|---|---|
| SD1 | 9.84 (COV = 3.25%) |
| SD2 | 9.94 (COV = 1.15%) |
| SD3 | 9.89 (COV = 0.39%) |

### 7.3 Drug Release Profile

Inhalable pharmaceuticals should possess adequate dissolution performance in order to activate drug absorption, especially when the fluid volume in the lungs is limited. Poorly soluble drugs are cleared either by the mucociliary escalator in the upper airway to the esophagus or by macrophage sequestration in the alveolar region (Riley et al., 2012). There is currently no pharmacopeial method for evaluating the dissolution performance of dry powder inhalation (“DPI”) formulations. One approach is to use a fast screening impactor (FSI) to separate the respirable fraction of the spray-dried particles, i.e., with MMAD<5 μm, prior to the dissolution test (Liao et al., 2019; Liao et al., 2021). After dispersion, the powders are deposited on a glass fiber filter and then transferred to a watch glass placed in the bottom of the dissolution chamber. This method, however, is not suitable for the present study. Spray drying of pure RDV produced fully amorphous content (FIG. 10). As amorphous materials confer inherent dissolution advantage, it is improper to compare the dissolution performance of spray-dried RDV−SA cocrystal with spray-dried pure RDV directly, as the actual improvement brought by cocrystallization would not be determined. On the other hand, the particle size of raw RDV in crystalline form is too large to be collected by the FSI. Comparing the dissolution performance of spray-dried RDV−SA cocrystal with raw RDV would not allow us to affirm whether the dissolution improvement is contributed to cocrystallization or solely the particle size reduction effect by spray drying. Considering these limitations, a dissolution test comparing raw RDV, RDV+SA physical mixture, and RDV−SA obtained from LAG was conducted in jacketed beakers containing 20 mL of SLF as dissolution medium. All samples were passed through a 63 μm sieve to minimize the variations in particle size and morphology. Although the test was not intended to be exhaustive, the findings could provide an insight on the dissolution of the samples for comparison purpose. As the average volume of lung fluid in human is around 0.37 mL/kg body weight (Vartak et al., 2021), 20 mL of SLF3 was employed in the test with an assumption for an individual with a body weight of 60 kg. The present disclosure adopted 10 mg of RDV (equivalent to 12.3 mg of RDV−SA) as the loaded dose in the NGI study based on the following justification. According to the prescribing information of the commercial intravenous RDV product VEKLURY, the recommended maintenance dose is 100 mg once daily (EMA, 2020b). Vermillion et al. demonstrated that a deposited dose of 0.35 mg/kg RDV in the lungs by inhalation in African green monkeys resulted in similar lung RDV triphosphate (the active metabolite of RDV) levels as 10 mg/kg intravenous RDV, demonstrating a dose-reduction of 10/0.35=˜28.5 times (Vermillion et al., 2021). Assuming similar pharmacokinetics and conversion efficiency of RDV into RDV triphosphate between African green monkeys and humans, a 100 mg dose of intravenous RDV in humans would require a deposited dose of ˜3.5 mg in the lungs by inhalation. Commercial DPI formulations inhaled using Breczhaler® result in 35% lung deposition (Weers et al., 2015), and hence a 10 mg loading dose of inhalable RDV DPI formulation would be sufficient to deposit ˜3.5 mg RDV into the lungs if the formulations have an FPF of at least 35%. In vitro aerosol performance testing confirmed that the FPF of RDV−SA SD2 and SD3 formulations were at least 35% (Table 5), inhalation of 12.3 mg RDV−SA would therefore be sufficient to deliver 3.5 mg RDV to the lungs and result in similar lung RDV triphosphate exposure relative to human IV doses of RDV.

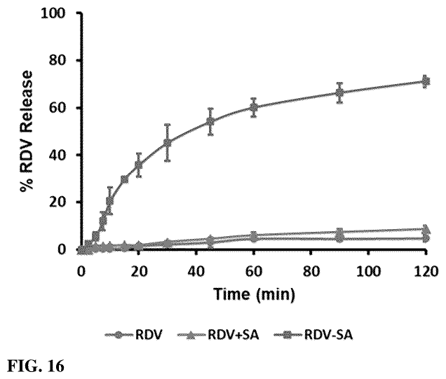

As illustrated in FIG. 16, the dissolution profile of the raw RDV and RDV+SA physical mixture plateaued at lower than 5% of release due to the low RDV aqueous solubility, whereas 71.22% RDV dissolved at 120 min for the cocrystal group. At the endpoint of the dissolution test RDV−SA displayed a 15.43-fold higher fraction of release at 120 min (p=0.0003). With a controlled particle size, it is believed that the dissolution improvement brought by cocrystallization was due to the solubility advantage offered by SA. According to Good and Rodriguez-Hornedo, the cocrystal solubility positively correlates with the coformer solubility, of which a coformer with a 10-fold higher solubility can usually lead to a cocrystal that is more soluble than the drug alone (RDV: 0.028 mg/mL, SA: 2.24 mg/mL in water at 25° C.) (Bernauer et al., 2018; Good and Rodriguez-Hornedo, 2009; Szente et al., 2021) further leading to an enhanced dissolution performance. As the spray-dried RDV−SA had a smaller particle size, it is reasonable to deduce that their superior dissolution performance would maintain.

### 7.4 Safety Profile

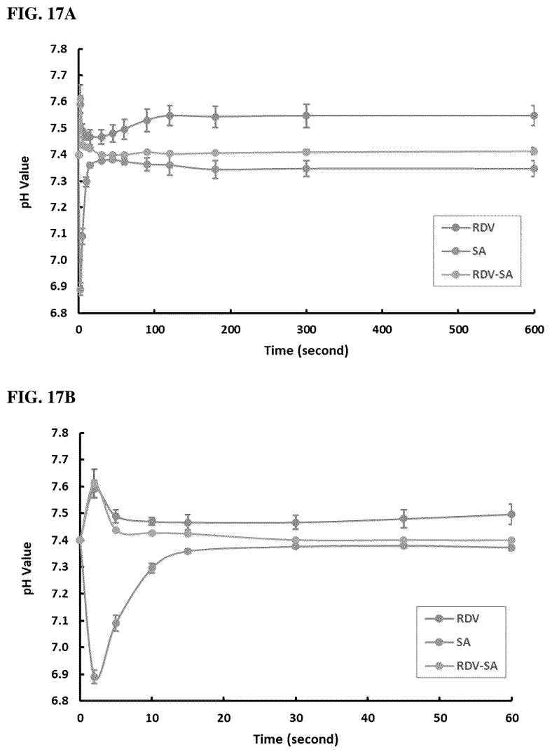

A major concern in formulating an inhalable RDV−SA cocrystal is potential respiratory irritation due to the acidic properties of SA. However, it should be noted that a very low respirable dose of RDV is already sufficient to exhibit antiviral effects in vivo (Vermillion et al., 2021). The 1:1 stoichiometry of RDV−SA therefore in turn implies a low dose requirement for SA, which may not necessarily induce drastic irritation or coughing. In this study, the pH change in response to the addition of RDV−SA into SLF was monitored as compared to pure RDV and SA. The real-time pH values against time during the dissolution were plotted in FIG. 17. The overall pH change between the RDV, SA, and RDV−SA groups did not show significant difference (p>0.05). The initial pH value of the dissolution medium, i.e., SLF, was measured to be 7.4. Pouring 0.017 mol of pure RDV into 20 mL SLF resulted in a slight increase of the medium pH to 7.55±0.04 in 10 min. Conversely, the addition of equimolar amount of pure SA into SLF separately caused a rapid but transient pH drop to 6.89±0.02 in 2 seconds due to its acidic property, followed by a gradual increase to reach a plateau from 15 seconds and achieving a final pH slightly below the original pH of SLF (7.35±0.03) at the end point of the dissolution. Notably, the RDV−SA cocrystal showed a more similar pH profile to pure RDV instead of SA. Despite the initial pH surge, it swiftly restored the original pH of SLF (7.41±0.01). Therefore, pH fluctuation in airway caused by inhalable RDV−SA is deemed minimal.

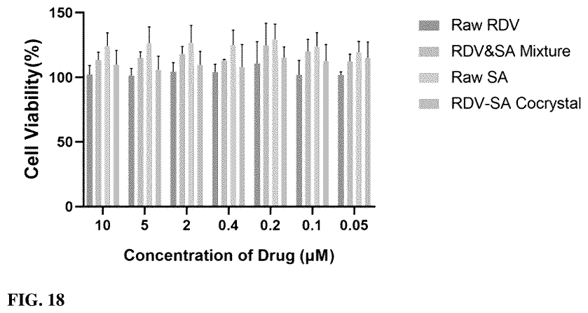

To further substantiate the safe use of RDV−SA for pulmonary delivery, the cytotoxicity of the inhalable RDV−SA cocrystal formulation was evaluated using the MTT cell viability assay in A549 cells, compared to the raw RDV, raw SA, and the RDV+SA physical mixture. A549 cells were used because it is one of the most studied human lung epithelial cell lines. In the concentration range of 0.05 to 10 μM, the cell viability for all groups were around 100% and no significant difference was found between RDV and RDV−SA groups (p>0.05, FIG. 18). Hence, RDV−SA is generally safe towards A549 cells without any in vitro cytotoxicity observed.

### Conclusion

As demonstrated herein, a new 1:1 cocrystal of RDV and SA was prepared from LAG. The clusiveness of the cocrystal is manifested by the thermally activated preparation method, where annealing at high temperature after grinding is essential to trigger the cocrystal growth. The use of different organic solvents as the cocrystallization medium in LAG generated different saturation levels, affecting the re-cocrystallization behavior of RDV−SA from an amorphous intermediate whereas the solvent effect was negated after annealing at high temperature. Compared to the raw RDV, the cocrystal displayed a superior dissolution performance in pH 7.4 simulated lung fluid and a good in vitro cytotoxicity profile in A549 cells. Spray drying was employed to reformulate the new RDV cocrystal for pulmonary delivery as inhalable dry powders, which can minimize the hepatic metabolism and directly target the infection site, potentially resulting in enhanced COVID-19 treatment. The optimized cocrystal formulation exhibited suitable MMAD and FPF for pulmonary delivery when dispersed at an inspiratory flow rate of 60 L/min. The favorable pharmaceutical properties of the cocrystal may be exploitable in the later formulation development and manufacturing of high-quality inhalable RDV products for clinical use. The present disclosure also highlights the importance of manipulating different annealing conditions during cocrystallization, particularly annealing temperature, in order to unveil more hidden cocrystals to facilitate the expansion of solid-state landscape.

### Exemplary Products, Systems and Methods Are Set Out in the Following Items

1. A cocrystal of remdesivir and salicylic acid.
2. The cocrystal according to item 1, belonging to a monoclinic P2₁ space group and having unit cell parameters a=18.3±0.1 Ang, b=5.6±0.1 Ang, c=19.3±0.1 Ang, α=γ=90°, β=112±1°.
3. The cocrystal according to item 1, which is characterized by a powder X-ray diffraction (PXRD) pattern measured using an incident beam of Cu Ka radiation comprising at least two X-ray diffraction peaks and the corresponding d-spacing values substantially as shown in Table 4.
4. The cocrystal according to item 1, which is characterized by a powder X-ray diffraction (PXRD) pattern measured using an incident beam of Cu Ka radiation comprising X-ray diffraction peaks below 20 degree two-theta substantially as shown in Table 4.
5. The cocrystal according to item 1, which is characterized by a powder X-ray diffraction (PXRD) pattern measured using an incident beam of Cu Ka radiation comprising X-ray diffraction peaks substantially as shown in Table 4.
6. The cocrystal according to item 1, which is characterized by a powder X-ray diffraction (PXRD) pattern measured using an incident beam of Cu Ka radiation substantially as shown in FIG. 7B (top five panels) or Table 4.
7. The cocrystal according to item 1, which is characterized by a Differential Scanning calorimetry (DSC) profile having a melting endotherm at about 138° C. to about 160° C., preferably at about 150° C. to about 155° C.
8. The cocrystal according to item 1, which is characterized by a Differential Scanning calorimetry (DSC) profile substantially as shown in FIG. 4B (top five panels) or FIG. 10.
9. The cocrystal according to item 1, which is characterized by Fourier-Transform Infrared Spectroscopy (FTIR) profile substantially as shown in FIG. 14.
10. The cocrystal according to item 1, which is prepared by a process comprising (a) liquid-assisted grinding; and (b) thermal annealing.
11. The cocrystal according to item 1, which is prepared by a process comprising (a) spray drying; and (b) thermal annealing.
12. A process for preparing a cocrystal of remdesivir and salicylic acid, the process comprising the steps of:
   (a) grinding remdesivir and salicylic acid in the presence of a solvent to produce a mixture; and
   (b) thermally annealing the mixture of step (a) at a temperature of at least about 60° C., preferably at least about 80° C.
13. A process for preparing a powder for inhalation, the powder comprising a cocrystal of remdesivir and salicylic acid, the process comprising the steps of
   (a) spray drying a solution of remdesivir and salicylic acid in a solvent to produce a mixture; and
   (b) thermally annealing the mixture of step (a) at a temperature of at least about 60° C., preferably at least about 80° C.
14. The process according to item 12 or 13, wherein the solvent is selected from the group consisting of acetonitrile, ethanol, methanol, acetone, and chloroform.
15. The process according to item 12 or 13, wherein the annealing is for a time period of about 30 minutes to about 120 minutes.
16. The process according to item 12 or 13, wherein the annealing is at a temperature of about 80° C. for a time period of about 30 minutes.
17. A pharmaceutical composition comprising as an active ingredient a remdesivir cocrystal according to any one of items 1 to 9.
18. The pharmaceutical composition according to item 17, wherein the composition is in a form of a powder for inhalation.
19. The pharmaceutical composition according to item 18, has a median mass aerodynamic diameter (MMAD) of below 5 microns, and a fine particle fraction (FPF) of at least about 30%.
20. The pharmaceutical composition according to item 18 or 19, wherein the powder for inhalation is excipient-free or comprises one or more excipients.
21. A method of treating a respiratory viral infection in a subject in need thereof, comprising administering to the respiratory system of the subject a composition comprising a therapeutically-effective amount of a cocrystal of remdesivir and salicylic acid.
22. The method of item 21, wherein the respiratory virus is a coronavirus.
23. The method of item 21, wherein the respiratory virus is selected from the group consisting of severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus.
24. The method of item 21, wherein the respiratory virus is SARS-COV-2 (COVID-19).
25. The method of item 21, wherein the composition is administered by oral inhalation.
26. The method of item 21, wherein the composition is administered by nasal inhalation.
27. The method of item 21, wherein the composition is administered with a dry powder inhaler.
28. A cocrystal of remdesivir and a coformer.
29. The cocrystal of item 28 wherein the remdesivir and a coformer have a molar ratio of 1:1.
30. The cocrystal according to item 29, wherein the coformer comprises at least one functional group selected from the group consisting of carboxylic acid, hydroxy, ether, aldehyde, ketone, ester, amide, amine, phosphonic acid, and sulfonic acid.
31. The cocrystal according to item 30, wherein the coformer comprises a benzoic acid derivative.
32. The cocrystal according to item 31, wherein the coformer is selected from the group consisting of salicylic acid, acetylsalicylic acid, and gentisic acid.
33. A method of treating a respiratory viral infection in a subject in need thereof, comprising administering to the respiratory system of the subject a composition comprising a therapeutically-effective amount of a cocrystal of remdesivir and a coformer.
34. The co-crystal according to item 1, characterized by an X-ray powder diffraction (XRPD) pattern having at least two X-ray diffraction peaks at 5.14±0.1, 5.62±0.1, 8.32±0.1, 15.00±0.1, 16.52±0.1, 18.58±0.1, and 21.48±0.1 degrees two theta (° q).
35. The co-crystal according to item 1, characterized by an X-ray powder diffraction (XRPD) pattern having X-ray diffraction peaks at 5.14±0.1, 5.62±0.1, 8.32±0.1, 15.00±0.1, 16.52±0.1, 18.58±0.1, and 21.48±0.1 degrees two theta (° q).

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following items and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

Abedi, F., Razavi, B. M., Hosseinzadeh, H., 2020. A review on gentisic acid as a plant derived phenolic acid and metabolite of aspirin: Comprehensive pharmacology, toxicology, and some pharmaceutical aspects. Phytotherapy research 34, 729-741.
Adams, C. J., Crawford, P. C., Orpen, A. G., Podesta, T. J., Salt, B., 2005. Thermal solid state synthesis of coordination complexes from hydrogen bonded precursors. Chem. Commun., 2457-2458.
Alhalaweh, A., Kaialy, W., Buckton, G., Gill, H., Nokhodchi, A., Velaga, S. P., 2013. Theophylline cocrystals prepared by spray drying: physicochemical properties and aerosolization performance. Aaps Pharmscitech 14, 265-276.
Altman, P., Wehbe, L., Dederichs, J., Guerin, T., Ament, B., Moronta, M. C., Pino, A. V., Goyal, P., 2018. Comparison of peak inspiratory flow rate via the Breezhaler®, Ellipta® and HandiHaler® dry powder inhalers in patients with moderate to very severe COPD: a randomized cross-over trial. BMC pulmonary medicine 18, 1-8.
Altomare, A., Cuocci, C., Giacovazzo, C., Moliterni, A., Rizzi, R., Corriero, N., Falcicchio, A., 2013. EXPO2013: a kit of tools for phasing crystal structures from powder data. J. Appl. Crystallogr. 46, 1231-1235.
Altomare, A., Giacovazzo, C., Guagliardi, A., Moliterni, A. G., Rizzi, R., Werner, P.-E., 2000. New techniques for indexing: N-TREOR in EXPO. J. Appl. Crystallogr. 33, 1180-1186.
Anandharamakrishnan, C., 2015. Spray drying techniques for food ingredient encapsulation. John Wiley & Sons.
Bennion, J. C., Matzger, A. J., 2021. Development and evolution of energetic cocrystals. Acc. Chem. Res. 54, 1699-1710.
Bennion, J. C., Siddiqi, Z. R., Matzger, A. J., 2017. A melt castable energetic cocrystal. Chem. Commun. 53, 6065-6068.
Bernauer, U., Bodin, L., Chaudhry, Q., Coenraads, P., Dusinska, M., Ezendam, J., Gaffet, E., Galli, C., Granum, B., Panteri, E., 2018. OPINION ON salicylic acid (CAS 69-72-7)-Submission I″ SCCS/1601/18-Preliminary opinion.
Bianchini, C., Peruzzini, M., Zanobini, F., 1991. Solid-state organometallic chemistry of tripodal (polyphosphine) metal complexes. Carbon-hydrogen activation reactions at cobalt (I) encapsulated into the tetraphosphine P (CH2CH2PPh2) 3. Organometallics 10, 3415-3417.
Bianconi, V., Violi, F., Fallarino, F., Pignatelli, P., Sahebkar, A., Pirro, M., 2020. Is acetylsalicylic acid a safe and potentially useful choice for adult patients with COVID-19? Drugs 80, 1383-1396.
Bis, J. A., Zaworotko, M. J., 2005. The 2-aminopyridinium-carboxylate supramolecular heterosynthon: A robust motif for generation of multiple-component crystals. Crystal growth & design 5, 1169-1179.
Bruni, G., Maietta, M., Maggi, L., Mustarelli, P., Ferrara, C., Berbenni, V., Milanese, C., Girella, A., Marini, A., 2013. Preparation and physicochemical characterization of acyclovir cocrystals with improved dissolution properties. J. Pharm. Sci. 102, 4079-4086.
Chen, J.-M., Li, S., Lu, T.-B., 2014. Pharmaceutical cocrystals of ribavirin with reduced release rates. Crystal growth & design 14, 6399-6408.
Chen, R., Weng, J., Chow, S. F., Lakerveld, R., 2020. Integrated Continuous Crystallization and Spray Drying of Insulin for Pulmonary Drug Delivery. Crystal Growth & Design.
Chidavaenzi, O. C., Buckton, G., Koosha, F., Pathak, R., 1997. The use of thermal techniques to assess the impact of feed concentration on the amorphous content and polymorphic forms present in spray dried lactose. Int. J. Pharm. 159, 67-74.
Chow, S. F., Chen, M., Shi, L., Chow, A. H., Sun, C. C., 2012. Simultaneously improving the mechanical properties, dissolution performance, and hygroscopicity of ibuprofen and flurbiprofen by cocrystallization with nicotinamide. Pharm. Res. 29, 1854-1865.
Chow, S. F., Shi, L., Ng, W. W., Leung, K. H. Y., Nagapudi, K., Sun, C. C., Chow, A. H., 2014. Kinetic entrapment of a hidden curcumin cocrystal with phloroglucinol. Crystal growth & design 14, 5079-5089.
Cui, W., He, Z., Zhang, Y., Fan, Q., Feng, N., 2019. Naringenin cocrystals prepared by solution crystallization method for improving bioavailability and anti-hyperlipidemia effects. AAPS PharmSciTech 20, 1-12.
Das, D., Langrish, T. A., 2012. Combined crystallization and drying in a pilot-scale spray dryer. Drying Technol. 30, 998-1007.
Évora, A. O., Castro, R. A., Maria, T. M., Rosado, M. T., Ramos Silva, M., Matos Beja, A., Canotilho, J., Eusébio, M. E. S., 2011. Pyrazinamide-diflunisal: a new dual-drug co-crystal. Crystal growth & design 11, 4780-4788.
Flament, M.-P., Leterme, P., Gayot, A., 2004. The influence of carrier roughness on adhesion, content uniformity and the in vitro deposition of terbutaline sulphate from dry powder inhalers. Int. J. Pharm. 275, 201-209.
Friščić, T., Childs, S. L., Rizvi, S. A., Jones, W., 2009. The role of solvent in mechanochemical and sonochemical cocrystal formation: a solubility-based approach for predicting cocrystallisation outcome. CrystEngComm 11, 418-426.
Friscic, T., Jones, W., 2009. Recent advances in understanding the mechanism of cocrystal formation via grinding. Cryst. Growth Des. 9, 1621-1637.
FrišČiĆ, T., Jones, W., 2011. Application of mechanochemistry in the synthesis and discovery of new pharmaceutical forms: co-crystals, salts and coordination compounds, Pharmaceutical Salts and Co-crystals, pp. 154-187.
Geiger, N., König, E.-M., Oberwinkler, H., Roll, V., Diesendorf, V., Faehr, S., Obernolte, H., Sewald, K., Wronski, S., Steinke, M., 2022. Acetylsalicylic acid and Salicylic acid inhibit SARS-COV-2 replication in precision-cut lung slices. bioRxiv.
Glatthaar-Saalmüller, B., Mair, K.H., Saalmüller, A., 2017. Antiviral activity of aspirin against RNA viruses of the respiratory tract—an in vitro study. Influenza and other respiratory viruses 11,85-92.
Gonnissen, Y., Verhoeven, E., Peeters, E., Remon, J. P., Vervaet, C., 2008. Coprocessing via spray drying as a formulation platform to improve the compactability of various drugs. European journal of pharmaceutics and biopharmaceutics 69, 320-334.
Good, D. J., Rodriguez-Hornedo, N., 2009. Solubility advantage of pharmaceutical cocrystals. Cryst. Growth Des. 9, 2252-2264.
Hadiwinoto, G. D., Lip Kwok, P. C., Lakerveld, R., 2018. A review on recent technologies for the manufacture of pulmonary drugs. Therapeutic delivery 9, 47-70.
Hamdy, M. M., Abdel Moneim, M. M., Kamal, M. F., 2021. Accelerated stability study of the ester prodrug remdesivir: Recently FDA-approved Covid-19 antiviral using reversed-phase-HPLC with fluorimetric and diode array detection. Biomed. Chromatogr. 35, e5212.
Humeniuk, R., Mathias, A., Cao, H., Osinusi, A., Shen, G., Chng, E., Ling, J., Vu, A., German, P., 2020. Safety, tolerability, and pharmacokinetics of remdesivir, an antiviral for treatment of COVID-19, in healthy subjects. Clinical and translational science 13, 896-906.
Jayasankar, A., Somwangthanaroj, A., Shao, Z. J., Rodríguez-Hornedo, N., 2006. Cocrystal formation during cogrinding and storage is mediated by amorphous phase. Pharm. Res. 23, 2381-2392.
Ji, X., Wu, D., Li, C., Li, J., Sun, Q., Chang, D., Yin, Q., Zhou, L., Xie, C., Gong, J., 2021. Enhanced Solubility, Dissolution, and Permeability of Abacavir by Salt and Cocrystal Formation. Crystal Growth & Design 22, 428-440.
Krupa, A., Descamps, M., Willart, J.-F., Strach, B., Wyska, E. b., Jachowicz, R., Danede, F., 2016. High-energy ball milling as green process to vitrify tadalafil and improve bioavailability. Molecular pharmaceutics 13, 3891-3902.
Kuroda, R., Yoshida, J., Nakamura, A., Nishikiori, S.-i., 2009. Annealing assisted mechanochemical syntheses of transition-metal coordination compounds and co-crystal formation. CrystEngComm 11, 427-432.
Labiris, N., Dolovich, M., 2003. Pulmonary drug delivery. Part I: physiological factors affecting therapeutic effectiveness of aerosolized medications. British journal of clinical pharmacology 56, 588-599.
Li, J., Zhang, K., Wu, D., Ren, L., Chu, X., Qin, C., Han, X., Hang, T., Xu, Y., Yang, L., 2021. Liposomal remdesivir inhalation solution for targeted lung delivery as a novel therapeutic approach for COVID-19. Asian journal of pharmaceutical sciences 16, 772-783.
Liang, W., Pan, H. W., Vllasaliu, D., Lam, J. K., 2020. Pulmonary Delivery of Biological Drugs. Pharmaceutics 12, 1025.
Liao, Q., Lam, I. C., Lin, H. H., Wan, L. T., Lo, J. C., Tai, W., Kwok, P. C., Lam, J. K., 2020. Effect of formulation and inhaler parameters on the dispersion of spray freeze dried voriconazole particles. Int. J. Pharm., 119444.
Liao, Q., Yip, L., Chow, M. Y., Chow, S. F., Chan, H.-K., Kwok, P. C., Lam, J. K., 2019. Porous and highly dispersible voriconazole dry powders produced by spray freeze drying for pulmonary delivery with efficient lung deposition. Int. J. Pharm. 560, 144-154.
Liao, Q., Yuan, S., Cao, J., Tang, K., Qiu, Y., Seow, H. C., Man, R. C. H., Shao, Z., Huang, Y., Liang, R., 2021. Inhaled Dry Powder Formulation of Tamibarotene, a Broad-Spectrum Antiviral against Respiratory Viruses Including SARS-COV-2 and Influenza Virus. Advanced Therapeutics, 2100059.
Lombard, J., le Roex, T., Haynes, D. A., 2020. Competition between Hydrogen and Halogen Bonds: The Effect of Solvent Volume. Crystal Growth & Design 20, 7384-7391.
Mahler, D. A., 2017. Peak inspiratory flow rate as a criterion for dry powder inhaler use in chronic obstructive pulmonary disease. Annals of the American Thoracic Society 14, 1103-1107.
Marques, M. R., Loebenberg, R., Almukainzi, M., 2011. Simulated biological fluids with possible application in dissolution testing. Dissolution Technol 18, 15-28.
Mínguez Espallargas, G., Brammer, L., van de Streck, J., Shankland, K., Florence, A. J., Adams, H., 2006. Reversible extrusion and uptake of HCl molecules by crystalline solids involving coordination bond cleavage and formation. Journal of the American Chemical Society 128, 9584-9585.
Mura, P., Cirri, M., Faucci, M., Gines-Dorado, J., Bettinetti, G., 2002. Investigation of the effects of grinding and co-grinding on physicochemical properties of glisentide. J. Pharm. Biomed. Anal. 30, 227-237.
Oguchi, T., Tozuka, Y., Hanawa, T., Mizutani, M., Sasaki, N., Limmatvapirat, S., Yamamoto, K., 2002. Elucidation of solid-state complexation in ground mixtures of cholic acid and guest compounds. Chem. Pharm. Bull. 50, 887-891.
Ohtake, S., Izutsu, K.-i., Lechuga-Ballesteros, D., 2020. Drying Technologies for Biotechnology and Pharmaceutical Applications. Wiley Online Library.
Pekar, K. B., Lefton, J. B., McConville, C. A., Burleson, J., Sethio, D., Kraka, E., Runcevski, T., 2021. Mechanosynthesis of a Coamorphous Formulation of Creatine with Citric Acid and Humidity-Mediated Transformation into a Cocrystal. Crystal Growth & Design 21, 1297-1306.
Riley, T., Christopher, D., Arp, J., Casazza, A., Colombani, A., Cooper, A., Dey, M., Maas, J., Mitchell, J., Reiners, M., 2012. Challenges with developing in vitro dissolution tests for orally inhaled products (OIPs). Aaps Pharmscitech 13, 978-989.
Sahakijpijarn, S., Moon, C., Koleng, J. J., Christensen, D. J., Williams Iii, R. O., 2020. Development of remdesivir as a dry powder for inhalation by thin film freezing. Pharmaceutics 12, 1002.
Santos, D., Maurício, A. C., Sencadas, V., Santos, J. D., Fernandes, M. H., Gomes, P. S., 2018. Spray drying: an overview. Biomaterials-Physics and Chemistry-New Edition, 9-35.
Sarkar, A., Rohani, S., 2015. Cocrystals of acyclovir with promising physicochemical properties. J. Pharm. Sci. 104, 98-105.
Sarmah, K. K., Rajbongshi, T., Bhuyan, A., Thakuria, R., 2019. Effect of solvent polarity in mechanochemistry: preparation of a conglomerate vs. racemate. Chem. Commun. 55, 10900-10903.
Sekharan, S., Liu, X., Yang, Z., Liu, X., Deng, L., Ruan, S., Abramov, Y., Sun, G., Li, S., Zhou, T., 2021. Selecting a stable solid form of remdesivir using microcrystal electron diffraction and crystal structure prediction. RSC advances 11, 17408-17412.
Singh, D. P., Moore, C. A., Gilliland, A., Carr, J. P., 2004. Activation of multiple antiviral defence mechanisms by salicylic acid. Molecular plant pathology 5, 57-63.
Stavropoulos, K., Johnston, S. C., Zhang, Y., Rao, B. G., Hurrey, M., Hurter, P., Topp, E. M., Kadiyala, I., 2015. Cocrystalline solids of telaprevir with enhanced oral absorption. J. Pharm. Sci. 104, 3343-3350.
Sun, D., 2020. Remdesivir for treatment of COVID-19: combination of pulmonary and IV administration may offer aditional benefit. The AAPS journal 22, 1-6.
Sundaralingam, M. t., Jensen, L., 1965. Refinement of the structure of salicylic acid. Acta crystallographica 18, 1053-1058.
Surov, A. O., Voronin, A. P., Manin, A. N., Manin, N. G., Kuzmina, L. G., Churakov, A. V., Perlovich, G. L., 2014. Pharmaceutical cocrystals of diflunisal and diclofenac with theophylline. Molecular pharmaceutics 11, 3707-3715.
Szente, L., Puskás, I., Sohajda, T., Varga, E., Vass, P., Nagy, Z. K., Farkas, A., Várnai, B., Béni, S., Hazai, E., 2021. Sulfobutylether-beta-cyclodextrin-enabled antiviral remdesivir: characterization of electrospun-and lyophilized formulations. Carbohydr. Polym. 264, 118011.
Tantry, U. S., Schror, K., Navarese, E. P., Jeong, Y.-H., Kubica, J., Bliden, K. P., Gurbel, P. A., 2021. Aspirin as an Adjunctive Pharmacologic Therapy Option for COVID-19: Anti-Inflammatory, Antithrombotic, and Antiviral Effects All in One Agent. Journal of Experimental Pharmacology 13, 957.
Tsuchiya, R., Uehara, A., Kyuno, E., 1974. SOLID-PHASE THERMAL SE REACTION OF METAL COMPLEXES. Chem. Lett. 3, 21-24.
Vartak, R., Patil, S. M., Saraswat, A., Patki, M., Kunda, N. K., Patel, K., 2021. Aerosolized nanoliposomal carrier of remdesivir: an effective alternative for COVID-19 treatment in vitro. Nanomedicine 16, 1187-1202.
Weers, J. G., Clark, A. R., Rao, N., Ung, K., Haynes, A., Khindri, S. K., Perry, S. A., Machineni, S., Colthorpe, P., 2015. In vitro-in vivo correlations observed with Indacaterol-based formulations delivered with the Breczhaler®. Journal of Acrosol Medicine and Pulmonary Drug Delivery 28, 268-280.
Vermillion, M. S., Murakami, E., Ma, B., Pitts, J., Tomkinson, A., Rautiola, D., Babusis, D., Irshad, H., Seigel, D., Kim, C., 2021. Inhaled remdesivir reduces viral burden in a nonhuman primate model of SARS-COV-2 infection. Science translational medicine 14, cab18282.
Weng, J., Wong, S. N., Xu, X., Xuan, B., Wang, C., Chen, R., Sun, C. C., Lakerveld, R., Kwok, P. C. L., Chow, S. F., 2019. Cocrystal engineering of itraconazole with suberic acid via rotary evaporation and spray drying. Crystal Growth & Design 19, 2736-2745.
Wong, S. N., Chan, S. W. S., Peng, X., Xuan, B., Lec, H. W., Tong, H. H., Chow, S. F., 2020. Effects of the Glass-Forming Ability and Annealing Conditions on Cocrystallization Behaviors via Rapid Solvent Removal: A Case Study of Voriconazole. Pharmaceutics 12, 1209.
Wong, S. N., Chen, Y. C. S., Xuan, B., Sun, C. C., Chow, S. F., 2021. Cocrystal Engineering of Pharmaceutical Solids: Therapeutic Potentials and Challenges. CrystEngComm 23, 7005-7038.
Wong, S. N., Hu, S., Ng, W. W., Xu, X., Lai, K. L., Lec, W. Y. T., Chow, A. H. L., Sun, C. C., Chow, S. F., 2018. Cocrystallization of curcumin with benzenediols and benzenetriols via rapid solvent removal. Crystal Growth & Design 18, 5534-5546.
Wong, S. N., Low, K.-H., Weng, J., Chan, H. W., Chow, S. F., 2022a. Expanding the solid-state landscape of dexamethasone: a specific sandwich structure in facilitating the formation of kinetically stable cocrystals from mechanochemistry. CrystEngComm.
Wong, S. N., Weng, J., Ip, I., Chen, R., Lakerveld, R., Telford, R., Blagden, N., Scowen, I. J., Chow, S. F., 2022b. Rational Development of a Carrier-Free Dry Powder Inhalation Formulation for Respiratory Viral Infections via Quality by Design: A Drug-Drug Cocrystal of Favipiravir and Theophylline. Pharmaceutics 14, 300.
Wood, P. A., Feeder, N., Furlow, M., Galek, P. T., Groom, C. R., Pidcock, E., 2014. Knowledge-based approaches to co-crystal design. CrystEngComm 16, 5839-5848.
Yonemochi, E., Hoshino, T., Yoshihashi, Y., Terada, K., 2005. Evaluation of the physical stability and local crystallization of amorphous terfenadine using XRD-DSC and micro-TA. Thermochim. Acta 432, 70-75.
Zhao, Q., Meng. M., Kumar, R., Wu, Y., Huang, J., Lian, N., Deng, Y., Lin, S., 2020. The impact of COPD and smoking history on the severity of COVID-19: a systemic review and meta-analysis. Journal of medical virology.

What is claimed is:

1. A cocrystal of remdesivir and salicylic acid.

2. The cocrystal according to claim 1, belonging to a monoclinic P2₁ space group and having unit cell parameters a=18.3±0.1 Ang, b=5.6±0.1 Ang, c=19.3±0.1 Ang, α=γ=90°, and β=112±1°.

3. The cocrystal according to claim 1, characterized by a powder X-ray diffraction (PXRD) pattern measured using an incident beam of Cu Ka radiation comprising at least two X-ray diffraction peaks and the corresponding d-spacing values substantially as shown in Table 4.

4. The cocrystal according to claim 1, characterized by a powder X-ray diffraction (PXRD) pattern measured using an incident beam of Cu Ka radiation comprising X-ray diffraction peaks below 20 degree two-theta substantially as shown in Table 4.

5. The cocrystal according to claim 1, is characterized by a powder X-ray diffraction (PXRD) pattern measured using an incident beam of Cu Ka radiation substantially as shown in FIG. 7B (top five panels) or Table 4.

6. The cocrystal according to claim 1, is characterized by a Differential Scanning calorimetry (DSC) profile having a melting endotherm at about 138° C. to about 160° C., preferably at about 150° C. to about 155° C.

7. The cocrystal according to claim 1, characterized by a Differential Scanning calorimetry (DSC) profile substantially as shown in FIG. 4B (top five panels) or FIG. 10.

8. The cocrystal according to claim 1, characterized by Fourier-Transform Infrared Spectroscopy (FTIR) profile substantially as shown in FIG. 14.

9. The cocrystal according to claim 1, which is prepared by a process comprising (a) liquid-assisted grinding; and (b) thermal annealing.

10. The cocrystal according to claim 1, which is prepared by a process comprising (a) spray drying; and (b) thermal annealing.

11. A process for preparing the cocrystal of remdesivir and salicylic acid according to claim 1, the process comprising the steps of:
(a) grinding remdesivir and salicylic acid in the presence of a solvent to produce a mixture; and
(b) thermally annealing the mixture of step (a) at a temperature of at least about 60° C., preferably at least about 80° C., for a time period of at least about 30 minutes.

12. A process for preparing a powder for inhalation, the powder comprising the cocrystal of remdesivir and salicylic acid according to claim 1, the process comprising the steps of
(a) spray drying a solution of remdesivir and salicylic acid in a solvent to produce a mixture; and
(b) thermally annealing the mixture of step (a) at a temperature of at least about 60° C., preferably at least about 80° C., for a time period of at least about 30 minutes.

13. A pharmaceutical composition comprising as an active ingredient a remdesivir cocrystal according to claim 1.

14. The pharmaceutical composition according to claim 13, wherein the composition is in a form of a powder for inhalation.

15. The pharmaceutical composition according to claim 14, has a median mass aerodynamic diameter (MMAD) of below 5 microns, and a fine particle fraction (FPF) of at least about 30%.

16. A method of treating a respiratory viral infection in a subject in need thereof, comprising administering to the respiratory system of the subject a composition comprising a therapeutically-effective amount of a cocrystal of remdesivir and salicylic acid according to claim 1.

17. The method of claim 16, wherein the respiratory virus is selected from the group consisting of a coronavirus, severe acute respiratory syndrome (SARS) coronavirus (SARS-COV), SARS-COV-2 (COVID-19), Middle East Respiratory Syndrome (MERS), respiratory syncytial virus (RSV), influenza virus, parainfluenza virus (PIV), pneumovirus (PMV), metapneumovirus (MPV), respirovirus, and rubulavirus.

18. The method of claim 16, wherein the composition is administered by oral inhalation, nasal inhalation, or with a dry powder inhaler (DPI).

19. A cocrystal of remdesivir and a coformer.

20. The cocrystal of claim 19, wherein the remdesivir and a coformer have a molar ratio of 1:1.

21. The cocrystal according to claim 19, wherein the coformer comprises at least one functional group selected from the group consisting of carboxylic acid, hydroxy, ether, aldehyde, ketone, ester, amide, amine, phosphonic acid, and sulfonic acid.

22. The cocrystal according to claim 21, wherein the coformer comprises a benzoic acid derivative.

23. The cocrystal according to claim 22, wherein the coformer is selected from the group consisting of salicylic acid, acetylsalicylic acid, and gentisic acid.

24. A method of treating a respiratory viral infection in a subject in need thereof, comprising administering to the respiratory system of the subject a composition comprising a therapeutically-effective amount of a cocrystal of remdesivir and a coformer according to claim 1.
